(19) United States
(12) Patent Application Publication
(10) Pub. No.: US 2026/0317780 A1
(43) Pub. Date: Oct. 15, 2026

(54) RETROVIRAL VECTOR PARTICLE PSEUDOTYPED WITH ENVELOPE PROTEINS OF CANINE DISTEMPER VIRUS

(71) Applicant: Miltenyi Biotec B.V. & Co. KG, Bergisch Gladbach (DE)

(72) Inventors: Nicole Cordes-Paulitz, Bergisch Gladbach (DE); Nora Winter, Bergisch Gladbach (DE); Shima Ferdos, Bergisch Gladbach (DE); Thomas Schaser, Bergisch Gladbach (DE)

(73) Assignee: Miltenyi Biotec B.V. & Co. KG, Bergisch Gladbach (DE)

(21) Appl. No.: 19/478,771

(22) PCT Filed: Apr. 26, 2024
(86) PCT No.: PCT/EP2024/061623

§ 371 Date: Oct. 24, 2025

(30) Foreign Application Priority Data
Apr. 27, 2023 (EP) 23170226.7

(51) Int. Cl. C12N 15/86; C07K 14/005; C07K 16/28

(52) U.S. Cl. CPC C12N 15/86; C07K 14/005; C07K 16/2812; C07K 16/2815; C07K 2317/622; C07K 2319/02; C12N 2740/13043; C12N 2740/13045; C12N 2740/15043; C12N 2740/15045; C12N 2760/18422; C12N 2810/859

(57) ABSTRACT

The present invention provides a pseudotyped retroviral vector particle, wherein said retroviral vector particle comprises a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family, b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV), and wherein said retroviral vector particle comprises at least one nucleic acid sequence encoding a transgene, and wherein said retroviral vector particle is a lentiviral or gammaretroviral vector particle.

### FIELD OF THE INVENTION

The present invention generally relates to the field of the generation of pseudotyped retroviral vector particles, in particular to the generation of pseudotyped retroviral vector particles with envelope proteins of canine distemper virus.

### BACKGROUND OF THE INVENTION

Gene delivery using retroviral vectors is a widely-used approach to correct defective genes and provide new functions to cells. However, due to the nature of the commonly used type of retroviral vectors, they are not selective by design, which reduces the safety and applicability of retroviral vectors for gene therapy.

Usually, retroviral vectors are pseudotyped with the envelope protein of the Vesicular Stomatitis Virus (VSV-G). This pseudotype transduces a broad range of cells including therapeutic relevant cell types like T cells, but requires pre-selection to exclude transduction of off-target cells. Moreover, VSV-G pseudotyped retroviral vectors require activation of T cells with stimulatory agents prior to transduction to reach sufficient transduction efficiencies (Amirache et al. (2014)). In contrast, high selectivity was seen with retargeted lentiviral vectors pseudotyped with measles virus envelope proteins (MV-LV) comprising a protein with fusion activity (F protein) and a protein with antigen-binding activity (H protein) that has been fused to a scFv (WO2008/037458A2). The broad application of this system has been tested for a variety of antigens in vitro but also in vivo (Anliker et al. (2010)). However, MV-LV can only be produced with low productivity limiting their therapeutic application. In addition, in vivo gene therapy applications are hampered by high seroprevalence of neutralizing antibodies against Measles virus in the general population. To overcome these challenges, lentiviral vectors may be pseudotyped with Nipah virus glycoproteins (NiV-LV), which was shown to increase the productivity by 10-100-fold (Bender et al (2016)). However, retargeting of NiV-LV is limited by the targeted antigen of choice, since they can only use cell surface proteins as receptors that bring them closely enough to the cell membrane. Canine distemper virus (CDV) is closely related to measles virus and was shown to be suitable for retargeting by fusion of scFvs similar to measles virus glycoproteins (Miest et al (2011), Bah et al (2020)). In addition, pseudotyping of oncolytic measles viruses with CDV glycoproteins was shown to overcome inhibition by neutralizing antibodies against measles (Bah et al (2020), Munoz-Alia et al (2021)). Moreover, pseudotyping of lentiviral vectors with Canine distemper virus was described, however T cell specific targeting was not shown (WO2022/182704 A1).

Therefore, there is a need in then art for an improved or alternative pseudotyped retroviral vector particle. To enable broad clinical application, novel pseudotyped retroviral vectors are need that may enable highly selective but also flexible targeting, that may provide a higher productivity and avoid neutralization in human serum.

### BRIEF DESCRIPTION OF THE INVENTION

The present invention makes use of the discovery that retroviral vector particles can be targeted to a specific cell type of interest by pseudotyping with engineered viral glycoproteins.

Surprisingly, it was found that retroviral vector particles such as lentiviral vector particles or gamma-retroviral vector particles efficiently transduce target cells, when they are pseudotyped with protein H and protein F envelope proteins of canine distemper virus (CDV) and the protein H is fused at its ectodomain to a polypeptide that specifically binds directly to a target antigen on a target cell or indirectly via an adapter to an antigen of a target cell. Compared to other state-of-the-art retroviral vectors e.g. pseudotyped with measles glycoproteins, a significantly higher productivity was found for CDV-pseudotyped retroviral vectors. Cytoplasmic-tail truncations of the CDV envelope glycoproteins were described not to improve efficiency of pseudotyping (Muñoz-Alia & Russell, 2021). Hence it was surprising to find that truncation of the cytoplasmic portion of the H glycoproteins from 14-34 amino acids and a truncation of the cytoplasmic domain of the F protein by 30 amino acids increases the productivity of the retroviral vector particles compared to non-modified glycoproteins.

T cells play a key role in cell-mediated immunity and are therefore an interesting target for adoptive immunotherapy such as chimeric antigen receptor (CAR) T cell immunotherapy. State-of-the-art CAR T manufacturing processes involve isolation of T cells from the human body, polyclonal activation, genetic modification with lentiviral vectors (LV) followed by an expansion phase. This complex process is time and cost intensive, but also results in a high degree of in vitro modification of the T cells. Decreasing the process complexity e.g. by omitting the activation step or by reducing the expansion phase therefore offers the potential to improve the cost efficiency of CAR T manufacture. In vivo generation of CAR T cells offers the potential to further reduce the complexity of manufacturing and to completely avoid in vitro manipulation of the T cells. As especially in-vivo applications require a safe and efficient gene transfer system, the retroviral vector particle provides a solution therefor. The inventors identified scFv sequences that are especially well suited for targeting of antigen CD4 or CD8 that are expressed on CD4+ T cells and CD8+ T cells with the retroviral vector particles as disclosed herein. Especially suited for in vitro as well as for in vivo transduction of CD4+ T cells and/or CD8+ T cells are a retroviral vector particle pseudotyped with envelope proteins of canine distemper virus as disclosed herein having antigen binding sequences, that are scFvs, comprising SEQ ID NO:4 (VL) and SEQ ID NO:5 (VH), if the specificity of said polypeptide is for the target antigen CD4, or comprising SEQ ID NO:7 (VL) and SEQ ID NO:8 (VH), if the specificity of said polypeptide is for the target antigen CD8.

### BRIEF DESCRIPTION OF THE DRAWINGS

FIG. 1: Principle of CDV pseudotyped retroviral vectors.

Retroviral vector particles can be pseudotyped with the H and F protein of canine distemper virus. Both envelope proteins are truncated in their cytoplasmic tail and the H protein is mutated to avoid binding to the natural receptor. To enable retargeting, the H protein is fused with a polypeptide specific for a target antigen (Direct-LV) or specific for a tag on an adapter molecule that mediates binding to the target antigen (Adapter-LV). While Direct-LV can bind to cells expressing the target antigen, Adapter-LV only binds to the cells in presence of a tagged adapter that is specific for the target antigen.

FIG. 2: Generation of lentiviral vectors pseudotyped with CDV envelope proteins.

Lentiviral vectors were generated by transient transfection of HEK-293T cells using plasmids encoding the envelope proteins for attachment (H) and fusion (F), helper plasmids encoding gag, pol and rev and the plasmid encoding the transgene (GFP). In order to optimize the retroviral vector productivity, particles were produced with plasmids encoding the full-length F protein (Fwt) or encoding a FcΔ30 with a truncated cytoplasmic domain. Plasmids encoding the full-length H protein (Hwt) or H proteins with a truncated cytoplasmic domain (HcΔ14-34) fused to an a-biotin scFv (SEQ ID NO:18) were compared. Upon harvest of the LV, the transfection efficiency was be determined by quantification of the transgene expression by flow cytometry. Functional titers were quantified by transducing biotinylated SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume. A Surface expression of the H protein is shown. For this, HEK293T cells were stained with fluorescently labeled biotin, followed by flow cytometry.

B Transfection efficiency of the transfer vector is shown, which was analyzed by quantification of GFP positive cells using flow cytometry.

C Titers of unconcentrated retroviral vector particles (HcΔ14-34 with Fwt or FcΔ30) are shown.

D Titers of concentrated retroviral vector particles (HcΔ24 or HcΔ30 with FcΔ30) are shown.

FIG. 3: Comparison of CDV and MV-pseudotyped LV.

Lentiviral vectors were generated by transient transfection of HEK-293T cells using plasmids encoding the envelope proteins for attachment (H) and fusion (F), helper plasmids encoding gag, pol and rev and the plasmid encoding the transgene (GFP). CDV-LVs were produced with plasmids encoding the full-length F protein (Fwt) or encoding a FcΔ30 with a truncated cytoplasmic domain. Plasmids encoding the full-length H protein (Hwt) or H proteins with a truncated cytoplasmic domain (HcΔ24-34) fused to an a-biotin scFv (SEQ ID NO:18) were compared. MV-LV were produced with plasmids encoding a FcΔ30 with a truncated cytoplasmic domain together with the H protein with a truncated cytoplasmic domain (HcΔ18) fused to an a-biotin scFv (SEQ ID NO:18). Upon harvest of the LV, the transfection efficiency was be determined by quantification of the transgene expression by flow cytometry. Functional titers were quantified by transducing biotinylated SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume.

A Surface expression of the H protein is shown. For this, HEK293T cells were stained with fluorescently labeled biotin, followed by flow cytometry.

B Transfection efficiency of the transfer vector is shown, which was analyzed by quantification of GFP positive cells using flow cytometry.

C Titers of unconcentrated retroviral vector particles (HcΔ24-34 with Fwt or FcΔ30) are shown.

D Titers of concentrated CDV-LV (HcΔ30 with FcΔ30) and MV-LV (HcΔ18 with FcΔ30) are shown.

FIG. 4: Generation of CDV-LVs with alternative truncations in H and F proteins.

Lentiviral vectors were generated by transient transfection of HEK-293T cells using plasmids encoding the envelope proteins for attachment (H) and fusion (F), helper plasmids encoding gag, pol and rev and the plasmid encoding the transgene (GFP). The LVs were produced with plasmids encoding the encoding a Fc Δ26, Fc Δ28, or FcΔ30 with a truncated cytoplasmic domain. Additional, N-terminally truncated F with unmodified cytoplasmic tail (SEQ ID NO:22) or FcΔ30 with truncated cytoplasmic tail (SEQ ID NO:23) were compared. Plasmids encoding the full-length H protein (Hwt) or H proteins with a truncated cytoplasmic domain (HcΔ20-34) fused to an a-biotin scFv (SEQ ID NO:18) were compared. Upon harvest of the LV, the transfection efficiency was be determined by quantification of the transgene expression by flow cytometry. Functional titers were quantified by transducing biotinylated SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume.

A Surface expression of the H protein is shown. For this, HEK293T cells were stained with fluorescently labeled biotin, followed by flow cytometry.

B Transfection efficiency of the transfer vector is shown, which was analyzed by quantification of GFP positive cells using flow cytometry.

C Titers of unconcentrated retroviral vector particles (HcΔ20-34 with FcΔ26, FcΔ28, FcΔ30, Fwt with N-terminal truncation or FcΔ30 with alternative additional N-terminal truncation) are shown.

FIG. 5: Transduction of PBMC with GFP encoding CDV-Adapter-LV.

Peripheral blood mononuclear cells (PBMC) were isolated from buffy coat of two healthy donors and activated overnight by cultivation in TexMACS™ supplemented with IL7, IL15 and TransAct™. The next day the cells were left untreated (UTD) or were transduced with a GFP encoding retroviral particle without adapter (w/o), or with biotinylated a-CD4 fab, a-CD4 f(ab)₂, a-CD8 fab or a-CD8 f(ab)₂ in presence (+) or absence (−) of Vectofusin-1® at a dose of 2.5 TU/cell. Retroviral vector particles with envelope proteins that are fused at its ectodomain to a polypeptide that specifically bind biotin (SEQ ID NO:18) were used. Transduction efficiency was analyzed five days post transduction, by staining for the targeted antigen and quantification of GFP-expressing cells using flow cytometry.

A Transduction efficiency of CD8+ T cells is shown.

B Transduction efficiency of CD4+ T cells is shown.

FIG. 6: Transduction of PBMC with CD20CAR encoding CDV-Adapter-LV.

Peripheral blood mononuclear cells (PBMC) were isolated from buffy coat of two healthy donors and activated overnight by cultivation in TexMACS™ supplemented with IL7, IL15 and TransAct™. The next day the cells were transduced with CD20CAR encoding retroviral particles at a dose of 0.5 TU/cell in presence of biotinylated a-CD4 fab, a-CD4 f(ab)₂, a-CD8 fab or a-CD8 f(ab)₂ with Vectofusin-1@. Retroviral vector particles with envelope proteins that are fused at the ectodomain to a biotin-binding polypeptide comprising either SEQ ID NO:18 (CDV-Bio3) or SEQ ID NO:21 (CDV-Bio2) were used. Transduction efficiency was analyzed six days post transduction, by staining for the targeted antigen and the transduction marker LNGFR, followed by quantification of LNGFR-expressing cells using flow cytometry.

A Transduction efficiency of CD8+ T cells is shown.

B Transduction efficiency of CD4+ T cells is shown.

FIG. 7: Generation of CAR-T cells with CDV-Adapter-LV Pan T cells of four healthy donors were isolated from buffy coat and activated over night by cultivation in TexMACS™ supplemented with IL7, IL15 and TransAct™. The next day, the cells were transduced with CD20CAR encoding retroviral vector particles at a dose of 0.5 TU/cell alone (w/o), in presence of either biotinylated a-CD4 f(ab)₂ or a-CD8 f(ab)₂ alone or in a mixture of both in a ratio of 1:1 or 5:1. Retroviral vector particles with envelope proteins that are fused at the ectodomain to a polypeptide that specifically binds biotin (SEQ ID NO:18) were used. Transduction efficiency was analyzed twelve days post transduction by staining for the targeted antigen and the transduction marker, followed by flow cytometry analysis.

A Transduction efficiency of CD8+ T cells is shown.

B Transduction efficiency of CD4+ T cells is shown.

FIG. 8: Surface expression of CDV-HcΔ30 fused to CD4 or CD8 specific scFv for the generation of direct-targeted CDV-LV HEK-293T cells were left untreated (mock) or were transfected with plasmids encoding CDV H proteins fused at the ectodomain to a polypeptide that specifically binds CD8 comprising SEQ ID NO: 9 or CD4 comprising SEQ ID NO: 24 or SEQ ID NO: 6. Surface expression of the CDV-HcΔ30 was determined by flow cytometry two days post transfection upon staining for the N-terminal HIS-tag.

A Surface expression of CDV-HcΔ30-CD8 (clone BW135) (SEQ ID NO: 9) and CDV-HcΔ30-CD4 (clone MT4-66) (SEQ ID NO: 24).

B Surface expression of CDV-HcΔ30-CD8 (clone BW135) (SEQ ID NO: 9) and CDV-HcΔ30-CD4 (VIT-4 or VIT-4-opt) (SEQ ID NO:6).

FIG. 9: Productivity of CD4 and CD8-specific direct-targeted CDV-LV Functional titers were quantified by transducing SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume.

A The titer of unconcentrated CDV-pseudotyped retroviral vector particles specific CD4 (SEQ ID NO: 6) and CD8 (SEQ ID NO: 9) are shown.

B The titer of concentrated CDV-pseudotyped retroviral vector particles specific CD4 (SEQ ID NO: 6) and CD8 (SEQ ID NO: 9) are shown.

FIG. 10:

Lentiviral vectors were generated by transient transfection of HEK-293T cells using plasmids encoding the envelope proteins for attachment (H) and fusion (F), helper plasmids encoding gag, pol and rev and the plasmid encoding the transgene (GFP). CDV-LVs were produced with plasmids encoding a FcΔ30 with a truncated cytoplasmic domain. Plasmids encoding the H proteins with a truncated cytoplasmic domain (HcΔ30) fused at the ectodomain to a CD8-binding polypeptide comprising SEQ ID NO: 9 or SEQ ID: 25 were compared. Upon harvest of the LV, the transfection efficiency was be determined by quantification of the transgene expression by flow cytometry. Functional titers were quantified by transducing SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume.

A Surface expression of the H protein is shown. For this, HEK293T cells were stained for the C-terminal HIS-tag of the scFV, followed by flow cytometry.

B Transfection efficiency of the transfer vector is shown, which was analyzed by quantification of GFP positive cells using flow cytometry.

C Titers of unconcentrated retroviral vector particles are shown.

FIG. 11: Transduction of PBMC with direct-targeted CDV-LV

Peripheral blood mononuclear cells (PBMC) were isolated from buffy coat of two healthy donors and activated overnight by cultivation in TexMACS™ supplemented with IL7, IL15 and TransAct™. The next day the cells were transduced with a GFP encoding retroviral particle at a dose of 1 TU/cell in presence (+) or absence (−) of Vectofusin-1®. Retroviral vector particles with envelope proteins that are fused at the ectodomain to a polypeptide that specifically binds CD4 (SEQ ID NO:6) (CDV-CD4) or CD8 (SEQ ID NO:9) (CDV-CD8) were used. Transduction efficiency was analyzed five days post transduction, by staining for the targeted antigen and quantification of GFP-expressing cells using flow cytometry.

A Transduction efficiency of CD8+ T cells is shown.

B Transduction efficiency of CD4+ T cells is shown.

FIG. 12: Generation of CAR-T cells with direct-targeted CDV-LV

Pan T cells of two healthy donors were isolated from buffy coat and activated over night by cultivation in TexMACS™ supplemented with IL7, IL15 and TransAct™. The next day, the cells were transduced with CD20CAR encoding retroviral vector particles at a dose of 1 TU/cell. Retroviral vector particles with envelope proteins that are fused at the ectodomain to a polypeptide that specifically binds CD4 (SEQ ID NO:6) (CDV-CD4) or CD8 (SEQ ID NO:9) (CDV-CD8) were used. Transduction efficiency was analyzed twelve days post transduction by staining for the targeted antigen and the transduction marker, followed by flow cytometry.

A Transduction efficiency of CD8+ T cells is shown.

B Transduction efficiency of CD4+ T cells is shown.

FIG. 13: Generation of CAR T cells in vivo.

NSG mice were transplanted with human PBMC by i.v. injection of 1×10⁷ cells per mouse. The next day, the mice were i.p. injected with 50 μg of adapter molecules when using αCD8- or αCD4-fab alone or 25 μg of each fab molecule when using a combination. About 2-3 h post adapter injection respective mice were i.v. injected with of CD8-specific, CD4-specific (Direct-LV) or biotin-specific CDV-LV (Adapter-LV) encoding a CD20CAR. CAR expressing T cells were quantified from blood on day 7 and 13 post transduction. On day 16-17 post transduction, the mice were sacrificed and blood, bone marrow and spleen tissue samples were analyzed by staining for transduced T cells using flow cytometry.

A Schematic description of the experimental layout for the in vivo generation of CAR T cells.

B Transduction efficiency of CD8+ and CD4+ T cells in blood over time is shown.

C Transduction efficiency of CD8+ T cells isolated from blood, bone marrow or spleen is shown.

D Transduction efficiency of CD4+ T cells isolated from blood, bone marrow or spleen is shown.

E The CD4/CD8 ratio of T cells isolated from blood, bone marrow or spleen is shown. Data represents mean±SD for n=6.

### DETAILED DESCRIPTION OF THE INVENTION

In a first aspect the present invention provides a pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV), and
wherein said retroviral vector particle comprises at least one nucleic acid sequence/molecule encoding a transgene, and wherein said retroviral vector particle is a lentiviral or gammaretroviral vector particle.

Said polypeptide that specifically binds to a target antigen expressed on the surface of a target cell may be a polypeptide comprising an antigen binding domain specific for a target antigen expressed on the surface of a target cell.

Said antigen binding domain (of an antibody) may be e.g. a Fab (fragment antigen binding), scFv (single chain fragment variable), single domain antibodies, diabodies, dsFv, Fab′, or single-chain antibody molecules.

Said pseudotyped retroviral vector particle as disclosed herein, wherein said Paramyxoviridae virus (of both the protein H and the envelope protein with fusion activity) is a virus of the morbillivirus genus, and wherein said virus of the morbillivirus genus is a canine distemper virus (CDV).

Preferentially the nucleic acid sequence/molecule encoding a transgene may be a nucleic acid sequence/molecule encoding a chimeric antigen receptor or a T cell receptor TCR. Said pseudotyped retroviral vector particle as disclosed herein, wherein said virus of the morbillivirus genus (of both the protein H and the envelope protein with fusion activity) is a canine distemper virus (CDV) or the 5804P strain or the Madrid/16 strain of the CDV.

Said pseudotyped retroviral vector particle, wherein said protein H of CDV lacks at least one part of the cytoplasmic region of said protein H, wherein said protein H is an N-terminal truncated protein H, and wherein said envelope protein with fusion activity (protein F) of CDV lacks at least one part of the cytoplasmic region of said protein F, wherein said protein F is a C-terminal truncated protein F.

In one embodiment of the invention said C-terminal truncated protein F comprises an N-terminal signal peptide sequence comprising SEQ ID NO:12.

In a preferred embodiment of the invention the pseudotyped retroviral vector particle is a pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
b) an envelope protein with fusion activity of a virus of the Paramyxoviridae family,
and wherein said Paramyxoviridae virus is a virus of the morbillivirus genus, and wherein said virus of the morbillivirus genus is a canine distemper virus (CDV), and wherein said retroviral vector particle comprises at least one nucleic acid molecule encoding a transgene, and wherein said retroviral vector particle is a lentiviral or gammaretroviral vector particle,
wherein said protein H of CDV lacks at least one part of the cytoplasmic region of said protein H, wherein said protein H is an N-terminal truncated protein H, and wherein said envelope protein with fusion activity (protein F) of CDV lacks at least one part of the cytoplasmic region of said protein F, wherein said protein F is a C-terminal truncated protein F, wherein the N-terminal signal peptide sequence comprising SEQ ID NO:12 is present in said truncated protein F.

The pseudotyped retroviral vector particle as disclosed herein, wherein said truncated protein H is HcΔ21-Δ32 as compared to the unmodified protein H set forth in SEQ ID NO:10, and wherein said truncated protein F is FcΔ26-Δ30 as compared to the unmodified protein F set forth in SEQ ID NO:11.

The pseudotyped retroviral vector particle as disclosed herein, wherein said truncated protein H is HcΔ21-Δ32 (as compared to the unmodified protein H set forth in SEQ ID NO:10), and wherein said truncated protein F is FcΔ30 (as compared to the unmodified protein F set forth in SEQ ID NO:11).

In a preferred embodiment of the invention said truncated protein H is HcΔ30.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions D526A, I527S, S528A, R529A, Y547A and T548A as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions V478L, L479D, T544S and T548A as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions S194I, V195R, V478L, L479D, T544S and T548A as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions Y525A, D526A and R529A as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions D526S, I527S, S528A and R529A as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions V478L, L479P, T544S and T548P as compared to the unmodified protein H set forth in SEQ ID NO:10.

Said pseudotyped retroviral vector particle, wherein said protein H is a N-terminal truncated protein H, and wherein said truncated protein H is HcΔ21-Δ32 (as compared to the unmodified protein H set forth in SEQ ID NO:10), and wherein said truncated protein H comprises the amino acid substitutions of positions S194I, V195R, V478L, L479P, T544S and T548P as compared to the unmodified protein H set forth in SEQ ID NO:10.

In a preferred embodiment of the invention said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions D526A, I527S, S528A, R529A, Y547A and T548A as compared to the unmodified protein H set forth in SEQ ID NO:10.

Said pseudotyped retroviral vector particle, wherein said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions S194I, V195R, V478L, L479D, T544S and T548A as compared to the unmodified protein H set forth in SEQ ID NO:10.

Said pseudotyped retroviral vector particle, wherein said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions Y525A, D526A and R529A as compared to the unmodified protein H set forth in SEQ ID NO:10.

Said pseudotyped retroviral vector particle, wherein said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions D526S, I527S, S528A and R529A as compared to the unmodified protein H set forth in SEQ ID NO:10.

Said pseudotyped retroviral vector particle, wherein said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions V478L, L479P, T544S and T548P as compared to the unmodified protein H set forth in SEQ ID NO:10.

Said pseudotyped retroviral vector particle, wherein said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions S194I, V195R, V478L, L479P, T544S and T548P as compared to the unmodified protein H set forth in SEQ ID NO:10.

Said pseudotyped retroviral vector particle, wherein the N-terminal signal peptide sequence comprising SEQ ID NO:12 is present in said truncated protein F.

Said pseudotyped retroviral vector particle, wherein said polypeptide of said envelope protein with antigen-binding activity that comprises an antigen binding domain specific for a target antigen expressed on the surface of a target cell is specific for the target antigen CD4 or CD8.

Said pseudotyped retroviral vector particle, wherein said polypeptide of said envelope protein with antigen-binding activity comprises an antigen binding domain comprising SEQ ID NO:4 (VL) and SEQ ID NO:5 (VH), preferentially in the order of sequence from N- to C-terminus VL-VH, if the specificity of said polypeptide is for the target antigen CD4, or an antigen binding domain comprising SEQ ID NO:7 (VL) and SEQ ID NO:8 (VH), if the specificity of said polypeptide is for the target antigen CD8, preferentially in the order of sequence from N- to C-terminus VL-VH.

In a preferred embodiment of the invention the pseudotyped retroviral vector particle is pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
b) an envelope protein with fusion activity of a virus of the Paramyxoviridae family,
and wherein said Paramyxoviridae virus is a virus of the morbillivirus genus, and wherein said virus of the morbillivirus genus is a canine distemper virus (CDV), and wherein said retroviral vector particle comprises at least one nucleic acid molecule encoding a transgene, and wherein said retroviral vector particle is a lentiviral or gammaretroviral vector particle,
wherein said protein H of CDV lacks at least one part of the cytoplasmic region of said protein H, wherein said protein H is an N-terminal truncated protein H, and wherein said envelope protein with fusion activity (protein F) of CDV lacks at least one part of the cytoplasmic region of said protein F,
wherein said polypeptide of said envelope protein with antigen-binding activity comprises an antigen binding domain comprising SEQ ID NO:4 (VL) and SEQ ID NO:5 (VH), preferentially in the order of sequence from N- to C-terminus VL-VH, if the specificity of said polypeptide is for the target antigen CD4, or an antigen binding domain comprising SEQ ID NO:7 (VL) and SEQ ID NO:8 (VH), if the specificity of said polypeptide is for the target antigen CD8, preferentially in the order of sequence from N- to C-terminus VL-VH.

Said pseudotyped retroviral vector particle, wherein said protein F is a C-terminal truncated protein F, wherein the N-terminal signal peptide sequence comprising SEQ ID NO:12 is present in said truncated protein F.

Said pseudotyped retroviral vector particle as disclosed herein, wherein said polypeptide comprising an antigen binding domain specific for said target antigen expressed on the surface of a T cell is specific for the antigen CD4 and comprises SEQ ID NO:6, or wherein said polypeptide comprising an antigen binding domain specific for a target antigen expressed on the surface of a T cell is specific for the antigen CD8 and comprises SEQ ID NO:9.

Said pseudotyped retroviral vector particle, wherein said polypeptide of said envelope protein with antigen-binding activity comprises an antigen binding domain comprising SEQ ID NO:4 and SEQ ID NO:5 (anti-CD4), if the specificity of said polypeptide is for the target antigen CD4, or an antigen binding domain comprising SEQ ID NO:7 and SEQ ID NO:8 (anti-CD8), if the specificity of said polypeptide is for the target antigen CD8, and wherein said pseudotyped retroviral vector particle comprises a modulating protein comprising a functional ectodomain comprising an antigen binding domain specific for the antigen CD3.

Said modulating protein may comprise:
I) said functional ectodomain
II) a transmembrane domain
III) a cytoplasmic domain.

Said cytoplasmic domain of said modulating protein may comprise a viral incorporation motif.

Said viral incorporation motif of said modulating protein may be the incorporation motif of gp41 (ENV of HIV), preferentially comprising SEQ ID NO:2.

Said transmembrane domain of said modulating protein may be e.g. the transmembrane domain of CD8, CD28 or PDGFR.

Said transmembrane domain of said modulating protein may be the transmembrane domain of CD8 comprising SEQ ID NO:3.

Said pseudotyped retroviral vector particle, wherein said antigen binding domain of said modulating protein specific for the antigen CD3 may comprise SEQ ID NO:1 (mut Oct3 clone).

Said pseudotyped retroviral vector particle as disclosed herein, wherein said at least one nucleic acid sequence/molecule encoding a transgene is a chimeric antigen receptor (CAR) specific for an antigen expressed on the surface of a second target cell such as a cancer cell, or wherein said at least one nucleic acid sequence/molecule encoding a transgene is a T cell receptor (TCR) with specificity for an antigen of a second target cell such as a cancer cell.

Said pseudotyped retroviral vector particle as disclosed herein, wherein said pseudotyped retroviral vector particle comprises at least a second nucleic acid sequence/molecule encoding at least a second transgene such as a cytokine such as IL7, IL15, or IL21.

In a further aspect the present invention provides a pseudotyped retroviral vector particle as disclosed herein for use in immunotherapy.

In a further aspect the present invention provides a pseudotyped retroviral vector particle as disclosed herein for use in treatment of a disease in a subject in need thereof.

The disease may be a cancer, an autoimmune disease or an infectious disease.

The transgene may be a CAR specific for a tumor associated antigen (TAA) or a tumor specific antigen (TSA) expressed on the surface of a cancer cell.

In another aspect the present invention provides a composition comprising
i) a pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV), and
ii) said tagged polypeptide, wherein said tagged polypeptide binds specifically to a target antigen expressed on the surface of an immune cell,
wherein said retroviral vector particle comprises at least one nucleic acid sequence/molecule encoding a transgene, and wherein said retroviral vector particle is a lentiviral or gammaretroviral vector particle.

Said polypeptide that specifically binds to a tag of a tagged polypeptide may be a polypeptide comprising an antigen binding domain specific for a tag of a tagged polypeptide.

Said composition, wherein said protein H of CDV lacks at least one part of the cytoplasmic region of said protein H, wherein said protein H is an N-terminal truncated protein H, and wherein said envelope protein with fusion activity (protein F) of CDV lacks at least one part of the cytoplasmic region of said protein F, wherein said protein F is a C-terminal truncated protein F.

In one embodiment of the invention said C-terminal truncated protein F comprises an N-terminal signal peptide sequence comprising SEQ ID NO:12.

Said composition, wherein said truncated protein H is HcΔ21-Δ32 as compared to the unmodified protein H set forth in SEQ ID NO:10, and wherein said truncated protein F is FcΔ26-Δ30 as compared to the unmodified protein F set forth in SEQ ID NO:11.

Said composition, wherein said truncated protein H is HcΔ21-Δ32 (as compared to the unmodified protein H set forth in SEQ ID NO:10), and wherein said truncated protein F is FcΔ30 (as compared to the unmodified protein F set forth in SEQ ID NO: 11).

In a preferred embodiment of the invention said truncated protein H is HcΔ30.

Said composition, wherein said truncated protein H is HcΔ21-Δ32 (as compared to the unmodified protein H set forth in SEQ ID NO:10), and wherein said truncated protein H comprises the amino acid substitutions of positions D526A, I527S, S528A, R529A, Y547A and T548A as compared to the unmodified protein H set forth in SEQ ID NO:10.

In a preferred embodiment of the invention said protein H is a N-terminal truncated protein H, and wherein said truncated protein H is HcΔ30 (as compared to the unmodified protein H set forth in SEQ ID NO:10), and wherein said truncated protein H comprises the amino acid substitutions of positions D526A, I527S, S528A, R529A, Y547A and T548A as compared to the unmodified protein H set forth in SEQ ID NO:10.

Said composition, wherein the N-terminal signal peptide sequence comprising SEQ ID NO:12 is present in said truncated protein F.

Said composition, wherein said tagged polypeptide that binds specifically to a target antigen expressed on the surface of an immune cell comprises an antigen binding domain specific for the target antigen CD4 or CD8.

Said composition, wherein said antigen binding domain of said tagged polypeptide comprises an antigen binding domain comprising SEQ ID NO:4 (VL) and SEQ ID NO:5 (VH), if the specificity of said polypeptide is for the target antigen CD4, or an antigen binding domain comprising SEQ ID NO:7 (VL) and SEQ ID NO:8 (VH), if the specificity of said polypeptide is for the target antigen CD8.

Said composition, wherein said tag of said tagged polypeptide may be e.g. a hapten or dextran or a peptide such as a neo-peptide.

Said composition, wherein said tag of said tagged polypeptide may be biotin (a hapten).

Said composition, wherein said tag of said tagged polypeptide may be biotin, and wherein said polypeptide that specifically binds to said tag of said tagged polypeptide may comprise an antigen binding domain comprising SEQ ID NO:16 (VL) and SEQ ID NO:17 (VH), preferentially in the order of sequence from N- to C-terminus VL-VH, or an antigen binding domain comprising SEQ ID NO:19 (VL) and SEQ ID NO:20 (VH), preferentially in the order of sequence from N- to C-terminus VL-VH.

Said composition, wherein said tag of said tagged polypeptide may be biotin, and wherein said polypeptide that specifically binds to said tag of said tagged polypeptide may comprise SEQ ID NO:18. or SEQ ID NO:21. Preferentially, said polypeptide that specifically binds to biotin of said biotinylated polypeptide may comprise SEQ ID NO:21.

Said composition, wherein said antigen binding domain of said tagged polypeptide comprises an antigen binding domain comprising SEQ ID NO:4 and SEQ ID NO:5, if the specificity of said polypeptide is for the target antigen CD4, or an antigen binding domain comprising SEQ ID NO:7 and SEQ ID NO:8, if the specificity of said polypeptide is for the target antigen CD8, and wherein said pseudotyped retroviral vector particle comprises a modulating protein comprising a functional ectodomain comprising an antigen binding domain specific for the antigen CD3.

Said composition, wherein said antigen binding domain of said modulating protein specific for the antigen CD3 comprises SEQ ID NO:1 (mut Oct3 clone).

Said composition, wherein said at least one nucleic acid sequence/molecule encoding a transgene is a chimeric antigen receptor (CAR) specific for an antigen expressed on the surface of a second target cell such as a cancer cell, or wherein said at least one nucleic acid encoding a transgene is a T cell receptor (TCR) with specificity for an antigen of a second target cell such as a cancer cell.

In another aspect the present invention provides a combination (of compositions) comprising
i) (a first composition comprising) a pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV), and
ii) (a second composition comprising) said tagged polypeptide, wherein said tagged polypeptide binds specifically to a target antigen expressed on the surface of an immune cell,
wherein said retroviral vector particle comprises at least one nucleic acid sequence/molecule encoding a transgene, and wherein said retroviral vector particle is a lentiviral or gammaretroviral vector particle.

In a further aspect the present invention provides a composition or a combination of compositions as disclosed herein for use in immunotherapy.

In a further aspect the present invention provides a composition or combination of compositions as disclosed herein for use in treatment of a disease in a subject in need thereof.

The disease may be a cancer, an autoimmune disease or an infectious disease.

The transgene may be a CAR specific for a tumor associated antigen (TAA) or a tumor specific antigen (TSA) expressed on the surface of a cancer cell.

In another aspect the present invention provides a pharmaceutical composition comprising
i) a pseudotyped retroviral vector particle comprising
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
b) an envelope protein with fusion activity of a virus of the Paramyxoviridae family,
and wherein said Paramyxoviridae virus is a virus of the morbillivirus genus, and wherein said virus of the morbillivirus genus is a canine distemper virus (CDV), and
wherein said retroviral vector particle comprises at least one nucleic acid sequence/molecule encoding a transgene, and wherein said retroviral vector particle is a lentiviral or gammaretroviral vector particle; and optionally
ii) a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art.

In another aspect the present invention provides a pharmaceutical composition comprising
i) a pseudotyped retroviral vector particle comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and
wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV),
wherein said retroviral vector particle comprises at least one nucleic acid sequence/molecule encoding a transgene, and wherein said retroviral vector particle is a lentiviral or gammaretroviral vector particle, and
ii) said tagged polypeptide, wherein said tagged polypeptide binds specifically to a target antigen expressed on the surface of an immune cell;
and optionally
c) a pharmaceutically acceptable carrier.

In a further aspect, the present invention provides an in-vitro method for the generation of a sample of genetically modified T cells comprising
a) providing a sample comprising T cells
b) genetic modification of the T cells by targeted transduction with a pseudotyped retroviral vector particle, wherein said retroviral vector particle comprises

### A)

i) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, wherein said target cell is a T cell, and wherein said envelope protein is protein H of a virus of the Paramyxoviridae family,
ii) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV),
and wherein said retroviral vector particle comprises at least one nucleic acid sequence/molecule encoding a transgene, and wherein said retroviral vector particle is a lentiviral or gammaretroviral vector particle, or

### B)

a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV),
and wherein said pseudotyped retroviral vector is in combination with said tagged polypeptide, wherein said tagged polypeptide binds specifically to a target antigen expressed on the surface of a T cell; and wherein said retroviral vector particle comprises at least one nucleic acid sequence/molecule encoding a transgene, and wherein said retroviral vector particle is a lentiviral or gammaretroviral vector particle, thereby generating a sample of genetically modified T cells.

Said in-vitro method, wherein said sample comprising T cells is prepared by centrifugation before said step of modification.

Said in-vitro method, wherein the number of T cells in said generated sample is less than 10-fold higher compared to the number of T cells in said sample comprising T cells.

Said in-vitro method, wherein said method does not comprise an expansion step.

Said in-vitro method, wherein said method is performed in less than 144 hours, less than 120 hours, less than 96 hours, less than 72 hours, less than 48 hours, less than 24 hours, less than 12 hours, less than 6 hours, less than 2 hours, less than hours, less than 30 minutes, or less than 15 minutes.

Said in-vitro method, wherein said method is performed in a closed system

Said in-vitro method, wherein said method is performed in a closed system, and wherein said method is an automated method.

In another aspect the present invention provides an in-vivo method for treating a disease in a subject in need thereof comprising administering to said subject a pseudotyped retroviral vector particle, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
b) an envelope protein with fusion activity of a virus of the Paramyxoviridae family,
and wherein said Paramyxoviridae virus is a virus of the morbillivirus genus, and wherein said virus of the morbillivirus genus is a canine distemper virus (CDV), and
wherein said retroviral vector particle comprises at least one nucleic acid sequence/molecule encoding a transgene, and wherein said retroviral vector particle is a lentiviral or gammaretroviral vector particle.

In another aspect the present invention provides an in-vivo method for treating a disease in a subject in need thereof comprising
i) administering to said subject a pseudotyped retroviral vector particle comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and
wherein said envelope protein is protein H of a virus of the Paramyxoviridae family,
b) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV), and
ii) administering to said subject said tagged polypeptide, wherein said tagged polypeptide binds specifically to a target antigen expressed on the surface of an immune cell,
wherein said retroviral vector particle comprises at least one nucleic acid sequence/molecule encoding a transgene, and wherein said retroviral vector particle is a lentiviral or gammaretroviral vector particle.

The administration of the pseudotyped retroviral vector particle to said subject may be performed before, simultaneously or after the administration of the tagged polypeptide.

In another aspect the present invention provides a plasmid vector system (a kit) for generation of a pseudotyped retroviral vector particle comprising
a) a nucleic acid sequence/molecule encoding the envelope for a pseudotyped retroviral vector particle, wherein said retroviral vector particle comprises
i) an envelope protein with antigen-binding activity, wherein said envelope protein is a recombinant protein that does not interact with at least one of its native receptors and is fused at its ectodomain to a polypeptide that specifically binds to a target antigen expressed on the surface of a target cell, and wherein said envelope protein is protein H of a virus of the Paramyxoviridae family,
ii) an envelope protein with fusion activity of a virus of the Paramyxoviridae family,
and wherein said Paramyxoviridae virus is a virus of the morbillivirus genus, and wherein said virus of the morbillivirus genus is a canine distemper virus (CDV),
c) a nucleic acid sequence/molecule encoding gag/pol of HIV-1,
d) a nucleic acid sequence/molecule encoding a transgene,
wherein said retroviral vector particle is a lentiviral or gammaretroviral vector particle.

In another aspect the present invention provides a plasmid vector system (a kit) for generation of a pseudotyped retroviral vector particle comprising
a) a nucleic acid sequence/molecule encoding the envelope for a pseudotyped retroviral vector particle, wherein said retroviral vector particle comprises
i) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
ii) an envelope protein with fusion activity of a virus of the Paramyxoviridae family, and wherein said Paramyxoviridae virus is a virus of the morbillivirus genus, and wherein said virus of the morbillivirus genus is a canine distemper virus (CDV),
b) a nucleic acid sequence/molecule encoding gag/pol of HIV-1
c) a nucleic acid sequence/molecule encoding a transgene,
wherein said pseudotyped retroviral vector, when expressed, is in combination with said tagged polypeptide, wherein said tagged polypeptide binds specifically to a target antigen expressed on the surface of a target cell,
wherein said retroviral vector particle is a lentiviral or gammaretroviral vector particle.

A pseudotyped retroviral vector particle as disclosed herein may be used to transduce cells in vivo at any effective dosage. In some embodiments, the viral particle is administered to a subject in vivo by application to the tissue, the organ or to the blood circulation of a subject in need of therapy.

In case of use of an adapter-based retroviral vector system as disclosed herein, the adapter (tagged polypeptide) may be administered to a subject in vivo—in addition to the viral particle—by application to the tissue, the organ or to the blood circulation of a subject in need of therapy, said administration of the said adapter may be before, simultaneous to or after the administration of said viral particle.

In some embodiments, the pseudotyped retroviral vector particle as disclosed herein may be administered via a route of parenteral, intravenous, intramuscular, subcutaneous, intratumoral, intraperitoneal, or intralymphatic administration. In some embodiments, the viral particle may be administered multiple times.

In one embodiment of the invention the pseudotyped retroviral vector particle as disclosed herein may be administered intratumorally to a subject and thereby transduces a T cell portion of the tumor-infiltrating lymphocytes at the tumor site.

In one embodiment of the invention the pseudotyped retroviral vector particle as disclosed herein may be administered intravenously to a subject, and thereby transduces the T cells in the circulatory blood system.

In one embodiment of the invention the pseudotyped retroviral vector particle as disclosed herein may be administered by intra lymph node injection to a subject, and thereby transduces the T cells in the lymph node.

In one embodiment of the invention the pseudotyped retroviral vector particle as disclosed herein may be administered by intra splenic injection to a subject, and thereby transduces the T cells in the spleen.

The pseudotyped retroviral vector particle as disclosed herein may also be delivered to a subject according to viral titer (TU/mL). The amount of the pseudotyped retroviral vector particle as disclosed herein directly injected may be determined by total TU and can vary based on both the volume that could be feasibly injected to the site and the type of tissue to be injected. In some embodiments, the viral titer delivered is about 1×10⁵ to 1×10⁶, about 1×10⁵ to 1×10⁷, 1×10⁵ to 1×10⁷, about 1×10⁶ to 1×10⁹, about 1×10⁷ to 1×10¹⁰, about 1×10⁷ to 1×10¹¹, or about 1×10⁹ to 1×10¹¹ TU.

In one embodiment of the invention the pseudotyped retroviral vector particle as disclosed herein may transduce T cells that are provided in a cell culture, and thereby may activate and transduce the T cells of the cell culture. The transduced T cells may be expanded to a therapeutically effective amount. The expanded T cells may be subsequently administered to a subject in need thereof.

In one embodiment of the invention the pseudotyped retroviral vector particle as disclosed herein may transduce T cells that are provided in a closed system that may be an automated manufacturing system, and thereby may transduce the T cells of the cell culture. The transduced T cells may be expanded to a therapeutically effective amount. The expanded T cells may be subsequently administered to a subject in need thereof.

The formulations and compositions of the present invention may comprise a combination of any number of the pseudotyped retroviral vector particle as disclosed herein, and optionally one or more additional pharmaceutical agents (polypeptides, polynucleotides, compounds etc.) formulated in pharmaceutically acceptable compositions for administration to a cell, tissue, organ, or a subject, either alone, or in combination with one or more other modalities of therapy.

In some embodiments, the one or more additional pharmaceutical agent further increases transduction efficiency of vectors.

In one embodiment of the invention the pseudotyped retroviral vector particle as disclosed herein may deliver a protein of interest that may be used for vaccination purposes or gene editing with, for example, Crispr/Cas.

In another embodiment of the invention the pseudotyped retroviral vector particle as disclosed herein may be integration-deficient.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

Retroviridae is a virus family with a single-stranded, diploid, positive-sense RNA genome that is reverse-transcribed into a DNA intermediate that is then incorporated into the host cell genome. Retroviridae-derived viruses are enveloped particles with a diameter of 80-120 nm. (Retro-/lenti-/gammaretro-) viral vectors are replication-deficient viral particles that are derived from the corresponding virus family. They contain Gag and Pol proteins, a single-stranded RNA genome and are usually pseudotyped with heterologous envelope proteins derived from other viruses. The RNA genome of said viral vectors do not contain any viral gene to produce viral progeny, but psi elements and LTRs that are required for efficient packing and reverse transcription into DNA. The DNA intermediate may contain a gene of interest under the control of a suitable promoter, for example, the CMV promoter and the gene of interest is expressed upon integration of said DNA into the genome of the host cell. The process of entering the host cell, delivering the RNA genome, integration and expression of the gene of interest is called transduction. The minimal requirements of a gammaretrovirus or lentivirus based viral vector has been well-described in the art.

In addition, integrase-deficient retroviral vectors (ID-RVs) have been developed that cannot integrate the retroviral vector genome in the host cell genome. ID-RVs are derived from conventional retroviral vectors but contain no or a mutated form of the retroviral integrase.

Upon entry into the host cell, the retroviral vector genome is reverse-transcribed in the cytoplasm, delivered into the nucleus, but not stably integrated into the host cell genome. ID-RVs are useful tools to express the gene of interest transiently. The definition of retroviral vectors and transduction also extents the integration-deficient retroviral vectors and its application.

Lentivirus is a genus of Retroviridae that cause chronic and deadly diseases characterized by long incubation periods, in the human and other mammalian species. The best-known lentivirus is the Human Immunodeficiency Virus (HIV), which can efficiently infect nondividing cells, so lentiviral derived retroviral vectors are one of the most efficient methods of gene delivery.

Gammaretroviridae is a genus of the Retroviridae family. Representative species are the murine leukemia virus (MLV) and the feline leukemia virus (FLV).

Paramyxoviridae is a family of viruses in the order of Mononegavirales. There are currently 49 species in this family, divided among 7 genera. Diseases associated with this virus family include measles, mumps, and respiratory tract infections. Members of this virus family are enveloped viruses with a non-segmented, negative-strand RNA genome of about 16 kb. Two membrane proteins with two distinct functions appear as spikes on the virion surface. The H/HN/G proteins mediate binding to the receptor at the cell surface.

Thus, the term “(virus) envelope protein(s) that have antigen binding activity” as used herein refers to protein(s) on the viral envelope that are responsible for binding to complementary receptors or antigens on the cell membrane of a target cell. For Paramyxoviridae H, HN or G proteins are virus envelope protein(s) that have antigen binding activity.

Upon binding the H/HN/G proteins change their conformation that induces a process called fusion helper function, leading to subsequent conformational changes within the F protein that is mediating the fusion of the viral and cellular membrane. The capsid and viral genome may now enter and infect or transduce the host cell.

The term “(virus) envelope proteins(s) that have fusion activity” as used herein refers to protein(s) that initiate fusion of viral and cellular membrane. For Paramyxoviridae F proteins refer to virus envelope protein(s) that have fusion activity.

The term “ectodomain” or “extracellular part/domain” as used herein refers to a domain of a membrane protein that extends into the extracellular space (the space outside a cell or virion).

The term “activation” as used herein refers to inducing physiological changes of a cell that increase target cell function, proliferation and/or differentiation.

The term “pseudotyping” or “pseudotyped” as used herein refers to a viral vector particle bearing envelope glycoproteins derived from other viruses having envelopes. The host range of the lentiviral vectors or viral vector particles of the present invention can thus be expanded or altered depending on the type of cell surface receptor used by the glycoprotein.

The term “modulating protein” of a retroviral vector particle as used herein refers to a protein that may modulate, e.g. activate a T cell. This modulation or specifically this activation may be due to the binding of an antigen binding domain of said modulating protein to stimulatory or co-stimulatory receptors of T cells, e.g. CD3 expressed on the surface of T cells.

To generate retroviral vectors the gag, pol and env proteins needed to assemble the vector particle are provided in trans by means of a packaging cell line, for example, HEK-293T. This is usually accomplished by transfection of the packaging cell line with one or more plasmids containing the gag, pol and env genes. For the generation of pseudotyped vectors, the env gene, originally derived from the same retrovirus as the gag and pol genes and as the RNA molecule or expression vector, is exchanged for the envelope protein(s) of a different enveloped virus. As an example, the F and H or HN or G protein of Paramyxoviridae is used.

Thus, an exemplary pseudotyped vector particle based on the HIV-1 retrovirus comprises the (1) HIV-1 Gag and Pol proteins, (2) an RNA molecule derived from the HIV-1 genome that may be used to generate a retroviral vector particle based on the HIV-1 genome lacking the gag, env, pol, tat, vif, vpr, vpu and nef genes, but still comprising the LTRs, the psi element and a CMV promoter followed by the gene to be transduced, for example, a gene for the GFP protein, and (3) the F and H proteins of measles virus, for example, in a truncated form.

The terms “native receptor” or “originally receptor” as used herein may be used interchangeably and refer to the receptor or antigen expressed on the cell surface of a cell that is bound by the naturally occurring virus envelope protein with antigen (receptor) binding activity. The native measles virus receptors are SLAM, nectin-4 and CD46. The native canine distemper virus receptors are SLAM and nectin-4. Nipahvirus envelope proteins use ephrin-B2 and ephrin-B3 as receptors for entry.

The term “one envelope protein with antigen-binding activity that does not interact with at least one of its native receptor(s)” as used herein means that said protein has reduced or ablated interaction with at least one receptor of a cell that is normally targeted by the virus having said protein as described elsewhere herein. Reduced interaction means that said truncated and/or mutated protein interacts with said at least one native receptor at least 50% less efficient, at least 60% less efficient, at least 70% less efficient, at least 80% less efficient, at least 90% less efficient, at least 95% less efficient, at least 99% less efficient compared to the non-mutated protein. Preferentially said protein does not interact anymore with said at least one of its native receptors. The interaction may be the binding of these two molecules to each other. The less efficient interaction may be a reduced affinity of said protein to its native receptor. Said envelope protein with antigen-binding activity may have more than one native receptors, then the reduction or ablation of interaction of one of these native receptors of said protein results in a reduced tropism of the vector particle. The more interactions of said protein with its native receptors are inhibited by mutation the more effective is the reduction of tropism of the vector particle.

In some cases it may be sufficient to inhibit the interaction of some but not all native receptors to said protein as the remaining interactions are not of relevance in the intended application or use of the retroviral vector particle as disclosed herein, e.g. when a native receptor is not expressed on any cell (target cells and non-target cells) in the environment of target cells that are intended to be transduced.

If an envelope protein with antigen-binding activity has more than 2 native receptors, e.g. 3 native receptors, then preferentially said protein does not interact with the majority of its native receptors, e.g. 2 from 3.

More preferentially, the envelope protein with antigen-binding activity does not interact with all of its native receptors.

The term “tropism” as used herein refers to the host range or specificity of a virus or retroviral vector. As used herein, the envelope protein with antigen-binding activity that is fused at its ectodomain to a polypeptide comprising an antigen binding domain defines the host range of the retroviral vector. For the adaptable retroviral vector system, the tagged polypeptide specific for antigen expressed on target cells defines the host range of the retroviral vector.

The term “target cell” as used herein refers to a cell which expresses an antigen (a marker) on its cell surface that should be recognized (bound) by the pseudotyped retroviral vector particle as disclosed herein or the tagged polypeptide of the adaptable system as disclosed herein. The target cell may be T cell, a primary T cell or a cell line derived from a T cell. The target cell may be a mammalian cell such as a murine cell, preferentially the target cell is a human cell.

A mutation that ablates interaction of canine distemper virus H protein with SLAM and Nectin 4 may be e.g. the point mutation at position D526, 1527, S528, R529; Y547 and T548 according to amino acid number of SEQ ID NO:10, wherein amino these amino acids are replaced with another amino acid and this mutation prevents or assists in preventing interaction of the H protein with SLAM and nectin-4 (Bah et al (2020); von Messing et al (2005)).

A pseudotyped retroviral vector particle “derived from”, for example, HIV-1, as used in the present invention, refers to a particle in which the genetic information for the RNA and/or the Gag and Pol proteins comprised by the vector particle originate from said retrovirus, in the above case, HIV-1. The original retroviral genome can comprise mutations, such as deletions, frame shift mutations and insertions.

The terms “cytoplasmic domain”, “cytoplasmic portion”, “cytoplasmic tail”, “cytoplasmic region”, “intracellular domain” or “endodomain”, as used in herein refer to the portion of the respective protein that is adjacent to the transmembrane domain of the protein and, if the protein is inserted into the membrane under physiological conditions, extends into the cytoplasm or in case of viral particles reaching into the intravirion side. Within Paramyxoviridae all envelope proteins with antigen-binding function are characterized to date as type II membrane proteins, meaning that the cytoplasmic domain is located at the N-terminus of the envelope protein.

In case of the cytoplasmic domain of the modulating protein, the cytoplasmic domain refers to the portion of the protein that reaches into the intravirion side. The portion of the protein that reaches into the intravirion side may comprise at least 1, at least 2, at least 3 or at least 4 amino acids. The cytoplasmic domain of the modulating protein may comprise a viral incorporation motif. Said incorporation motif may be the incorporation motif of gp41.

The term “wherein said pseudotyped retroviral vector particle comprises at least a second nucleic acid sequence/molecule” refers to the protein expression of more than one protein from the same viral genome. This may for example be achieved by incorporation of a second open reading frame using an additional promotor, or by incorporation of viral motifs that allow the expression of two or more proteins from one transcript e.g. by using a P2A site.

The term “truncated”, as used in the present invention, refers to a deletion of amino acid residues of the designated protein. It is clear to the skilled person that a protein is encoded by a nucleic acid. Thus, “truncated” also refers to the corresponding coding nucleic acids in a nucleic acid molecule that codes for a given “truncated” protein.

In the present invention, specific reference is made to “truncated H”, or “truncated F” proteins, which designates the Paramyxoviridae, preferably Canine distemper virus H protein, F proteins, respectively, whose cytoplasmic portion has been partly or completely truncated, i.e. amino acid residues (or coding nucleic acids of the corresponding nucleic acid molecule encoding the protein) have been deleted.

The cytoplasmic portion of the F protein (termed Fc) is located at the C-terminus of the protein.

For all envelope proteins with the cytoplasmic portion located at the C-terminus one begins counting from the C-terminal end of the protein when ascertaining the desired sequence. As an example for Canine distemper virus strain 5840P F glycoprotein, FcΔ30 would refer to an F protein having deleted the last 30 amino acids counting from the C-terminal end of the protein F set forth in SEQ ID NO:11. Consequently, for the F protein derived from Canine distemper virus strain 5840P FcΔ30 would refer to an F protein having a cytoplasmic domain comprising amino acid sequence of SEQ ID NO:13.

By contrast, the cytoplasmic portion of the H, HN or G protein is located at the N-terminus (termed Hc).

Thus, one begins counting at the second amino acid residue of the N-terminal end of the H, HN or G protein (i.e. omitting the first methionine residue) when ascertaining the desired sequence.

The term “truncated protein H is HcΔ21-Δ32” in the context of the protein H of a CDV as used herein refers to any truncated protein H of the CDV having deleted the first 21 to 32 amino acids counting from the N-terminal end of the protein H set forth in SEQ ID NO:10 (the unmodified protein H): individually said truncated protein H may be:

HcΔ21, HcΔ22, HcΔ23, HcΔ24, HcΔ25, HcΔ26, HcΔ27, HcΔ28, HcΔ29, HcΔ30, HcΔ31 or HcΔ32.

As an example, for the H protein derived from Canine Distemper virus strain 5840P the cytoplasmic domain of HcΔ21 comprises the amino acids as in SEQ ID NO:14. Accordingly, the H protein derived from Canine Distemper virus strain 5840P the cytoplasmic domain of HcΔ30 comprises the amino acids as in SEQ ID NO:15.

Modifications that allow truncation for efficient pseudotyping may be combined with modifications that ablate native receptor binding function.

The person skilled in the art will readily be able to introduce mutations as, for example, additions and deletions, into a given nucleic acid or amino acid sequence.

The proteins of the present invention further include functional homologs. A protein is considered a functional homolog of another protein for a particular function, if the homolog has a similar function as the original protein. The homolog can be, for example, a fragment of the protein, or a substitution, addition, or deletion mutant of the protein.

Determining whether two amino acid sequences are substantially homologous is typically based on FASTA searches. For example, the amino acid sequence of a first protein is considered to be homologous to that of a second protein if the amino acid sequence of the first protein shares at least about 70% amino acid sequence identity, preferably at least about 80% identity, and more preferably at least about 85%, 90%, 95% or 99% identity, with the sequence of the second protein.

The terms “Psi positive” and “psi negative”, as used in the present application, refer to a nucleic acid molecule where the retroviral psi element is present and absent, respectively. The psi element is a cis-acting signal located near the 5′ end of the retroviral genome and designates a packaging signal, which is of importance during assembly of the viruses and leads to the incorporation of the viral RNA into the viral core. Thus, a psi negative RNA does not comprise the retroviral psi element and consequently will not be assembled into a vector particle of the present invention; in contrast, a psi positive RNA that does comprise said psi element will be effectively assembled into the vector particle.

The term “antigen expressed on the surface of a (target) cell” or “cell (surface) marker”, as used in the present invention, refers to a molecule present on the surface of a cell, preferentially on a target cell. Such molecules can be, inter alia, peptides or proteins that may comprise sugar chains or lipids, clusters of differentiation (CDs), antibodies or receptors. Since not all populations of cells express the same cell markers, a cell marker can thus be used to identify, select or isolate a given population of cells expressing a specific cell marker. As an example, CD4 is a cell marker expressed by T helper cells, regulatory T cells, and monocytes. Thus, T helper cells, regulatory T cells, and monocytes can be identified, selected or otherwise isolated, inter alia by a FACS cell sorter, by means of the CD4 cell marker.

The term “tagged polypeptide” that is specific for binding to a target antigen expressed on the surface of a T cell as used herein refers to a polypeptide that has bound thereto directly or indirectly at least one additional component, i.e. the tag. The tagged polypeptide as used herein is able to bind a target antigen expressed on T cells, regularly CD4 or CD8. The polypeptide may be an antibody or antigen binding fragment thereof that binds to said antigen expressed on the surface of a target cell. The polypeptide of the tagged polypeptide alternatively may be a cytokine or a growth factor or another soluble polypeptide that is capable of binding to an antigen of a target cell.

The term “adapter” or “adapter molecule” as used herein refers to a tagged polypeptide that can bind to a target antigen of a cell such as a T cell, e.g. antibody or antigen binding fragment thereof such as a scFv, and has bound thereto directly or indirectly at least one additional component, i.e. the tag. The adapter or adapter molecule may by a tagged antibody or antigen binding fragment thereof, a cytokine or a growth factor or another soluble polypeptide that is capable of binding to a target antigen of a T cell. The retroviral vector particle specific for a tag as disclosed herein may bind to said adapter.

Such an adaptable retroviral vector system, that comprises a pseudotyped retroviral vector particle specific for a tag and said tagged polypeptide specific for an antigen expressed on the surface of a target cell is disclosed e.g. in WO2019086351A1.

The tag of said tagged polypeptide may be e.g. a hapten or dextran and the hapten or dextran may be bound by the antigen binding domain of the polypeptide comprising an antigen binding domain specific for the tag. Said tag also may be a peptide such as a neo-peptide.

Haptens such as e.g. FITC, biotin, PE, streptavidin, thiamin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

The term “polypeptide comprising an antigen binding domain specific for a tag” as used herein refers to a polypeptide that can bind a tag of a tagged polypeptide. The tagged polypeptide is different from the polypeptide that comprises the antigen binding domain specific for the tag.

The polypeptide comprising the antigen binding domain specific for a tag may be an antibody or antigen binding fragment thereof that binds to said tag of the tagged polypeptide.

The term “polypeptide comprising an antigen binding domain specific for a tag” as used herein refers to a polypeptide that can bind a tag of a tagged polypeptide. The polypeptide comprising the antigen binding domain specific for a tag may be an antibody or antigen binding fragment thereof such as a scFv or a nanobody that binds to said tag of the tagged polypeptide.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”). Examples of antigen binding fragments include Fab (fragment antigen binding), scFv (single chain fragment variable), single domain antibodies, diabodies, dsFv, Fab′, single-chain antibody molecules, and multispecific antibodies formed from antibody fragments.

As used herein, the term “antigen” is intended to include substances that bind to or evoke the production of one or more antibodies and may comprise, but is not limited to, proteins, peptides, polypeptides, oligopeptides, lipids, carbohydrates such as dextran, and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “expression” as used herein is defined as the transcription and/or translation of a particular nucleotide sequence driven by its promoter in a cell.

As used herein, the term “subject” refers to an animal. Preferentially, the subject is a mammal such as mouse, rat, cow, pig, goat, chicken dog, monkey or human. More preferentially, the individual is a human. The subject may be a subject suffering from a disease such as cancer, an autoimmune disease or an infectious disease.

The term “administering” refers to local and systemic administration, e.g., including enteral, parenteral, pulmonary, and topical/transdermal administration. The administration may be directly intratumoral. Routes of administration for pharmaceutical ingredients include, e.g., oral administration, nasal or inhalation administration, administration as a suppository, topical contact, transdermal delivery, intrathecal administration, intravenous administration, intraperitoneal administration, intramuscular administration, intralesional administration, or subcutaneous administration to a subject. Administration can be by any route including parenteral and transmucosal (e.g, oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial.

A recombinant protein is a biotechnologically generated protein that does not occur naturally in a eukaryotic and/or prokaryotic cell. Often it is composed of different domains from different proteins, e.g. as used herein, a viral envelope protein is fused (at its ectodomain) to a polypeptide that comprises an antigen binding domain specific for an antigen or for a tag.

The terms “having specificity for”, “specifically binds” or “specific for” with respect to an antigen-binding domain of an antibody or a fragment thereof refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based cytotoxic responses to attack cancer cells. T cells that have a natural or genetically engineered reactivity to a patient's cancer are generated in-vitro and then transferred back into the cancer patient or are directly generated in-vivo. Then the immunotherapy is referred to as “CAR T cell immunotherapy”.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

The terms “engineered cell” and “genetically modified cell” as used herein can be used interchangeably. The terms mean containing and/or expressing a foreign gene or nucleic acid sequence/molecule which in turn modifies the genotype or phenotype of the cell or its progeny.

Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® (Miltenyi Biotec).

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature-controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising T cells may be provided in transfer bags or other suited containers which can be connected to the closed system by sterile means.

The term “providing a (cell) sample comprising T cells” means the provision of a cell sample, preferentially of a human cell sample of hematologic origin. Normally, the cell sample may be composed of hematologic cells from a donor or a patient. Such blood product can be in the form of whole blood, buffy coat, leukapheresis, PBMCs or any clinical sampling of blood product.

It may be from fresh or frozen origin.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

In general, T cells may be characterized based on their function and marker expression. Two main subgroups have been defined: CD4 expressing T cells (i.e. T helper cells) and CD8 expressing T cells (i.e. cytotoxic T cells). CD8 positive specifically lyse e.g. virus infected or tumor cells by releasing perforin, granzyme and FasL upon specific binding to the respective peptide presented on the MHC I to the TCR. On CD4+ T cells peptides presented on MHC II are bound specifically by the respective TCR inducing a signaling cascade triggering the release of several cytokines such as interferons and interleukins. Such cytokines may recruit other immune cells and may activate CD8+ T cells for a boosted and sustained cytolytic activity.

T cells differentiate into different phenotypes showing a specific memory or effector function profile.

Naive T cells (T_{N}) have recently undergone positive and negative selection in the thymus and are considered to be early differentiated with high memory function but a low effector function. They can be identified by flow cytometry expressing CD45RA, CCR7 and CD62L and being negative for CD45RO, CD95 and IL-2Rbeta. Naïve T cells in the blood are normally found in a quiescent state, which is characterized by small cell size, low proliferative capacity, low basal metabolic programs and low responsiveness to key cytokines, e.g. IL-2. The terms resting T cells”, “quiescent T cells”, “unstimulated T cells” and “non-activated T cells” may be used interchangeably.

Stem cell memory T cells (T_{SCM}) have a high potential for self-renewal, are minimally differentiated and can differentiate into other phenotypes. They can be identified by flow cytometry expressing CD45RA, CD45RO, CCR7, CD62L, CD95 and IL-2Rbeta.

Central memory T cells (T_{CM}) are characterized by a low effector function profile and a long persistence. Upon antigen encounter, this T cell subset expands rapidly and differentiate into T cells with effector function. They can be identified by flow cytometry expressing CD45RO, CCR7, CD62L, CD95 and IL-2Rbeta.

Effector memory T cells (T_{EM}) migrate to inflamed tissues and have an intermediate level of effector function. They can be identified by flow cytometry expressing CD45RO, CD95, IL-2Rbeta and being negative for CCR7 and CD62L.

Effector T cells (T_{EFF}) are short lived T cells with no memory function but the highest potential of cytolytic effector function. They can be identified by flow cytometry expressing CD45RA, CD95, IL-2Rbeta and being negative for CD45RO, CCR7 and CD62L.

In general, a chimeric antigen receptor (CAR) may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments the CAR may be an adaptable CAR system (similar to the adaptable retroviral vector system) and may be then referred to as “anti-tag” CAR or “adapterCAR” or “universal CAR” as disclosed e.g. in U.S. Pat. No. 9,233,125B2.

A “signal peptide” refers to a peptide sequence that directs the transport and localization of the protein within a cell during or post translation, e.g. to a certain cell organelle (such as the endoplasmic reticulum) and/or the cell surface.

Generally, an “antigen binding domain” of a CAR refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain of the CAR may comprise an antibody or an antigen binding fragment thereof. The antigen binding domain of the CAR may comprise, for example, full length heavy chain, Fab fragments, single chain Fv (scFv) fragments, nanobodies, divalent single chain antibodies or diabodies. Any molecule that binds specifically to a given antigen such as affibodies or ligand binding domains from naturally occurring receptors may be used as an antigen binding domain. Often the antigen binding domain of a CAR is a scFv. Normally, in a scFv the variable regions of an immunoglobulin heavy chain and light chain are fused by a flexible linker to form a scFv. Such a linker may be for example the “(G₄S)₃-linker”.

In some instances, it is beneficial for the antigen binding domain of the CAR to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain of the CAR and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. Splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

If the CAR is an inhibitory CAR (referred to normally as “iCAR”), then said CAR may have the same extracellular and/or transmembrane domains as the activating CAR but differs from the activating CAR with regard to the endodmain.

The at least one endodomain of the inhibitory CAR may be a cytoplasmic signaling domain comprising at least one signal transduction element that inhibits an immune cell or comprising at least one element that induces apoptosis.

The CARs that may be transduced by the pseudotyped retroviral vector particle as disclosed herein present may be designed to comprise any portion or part of the above-mentioned domains as described herein in any order and/or combination resulting in a functional CAR.

The term “display” as used herein refers to a protein or peptide that is incorporated into the viral envelope, thereby presenting the extracellular domain outside the viral particle.

### EXAMPLES

The following examples are intended for a more detailed explanation of the invention but without restricting the invention to these examples.

### Example 1: CDV Pseudotyped Retroviral Vector System

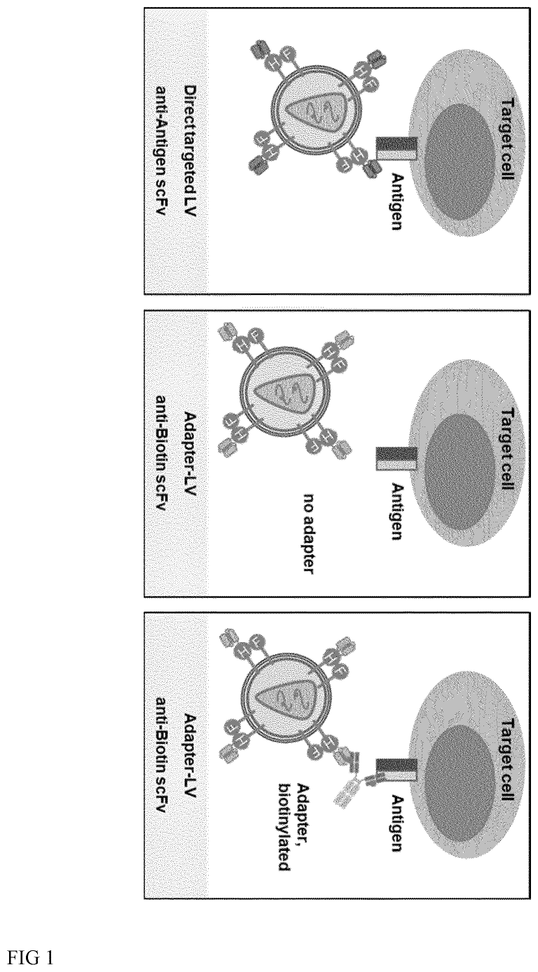

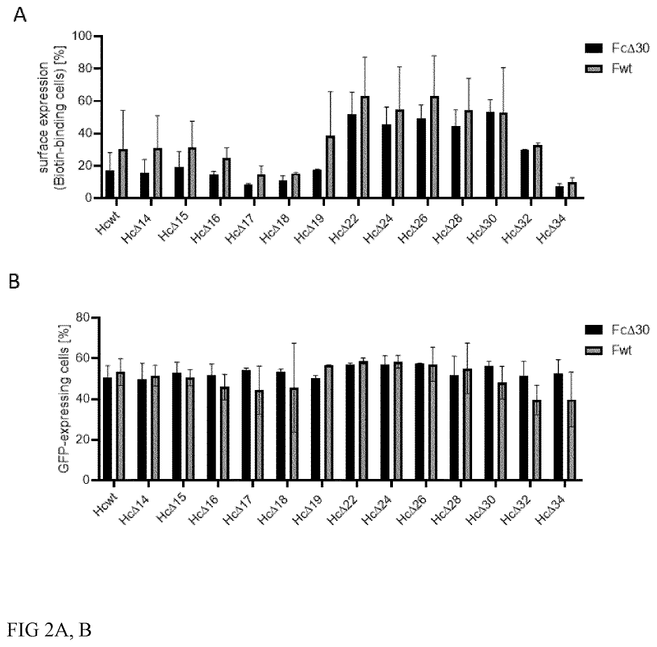

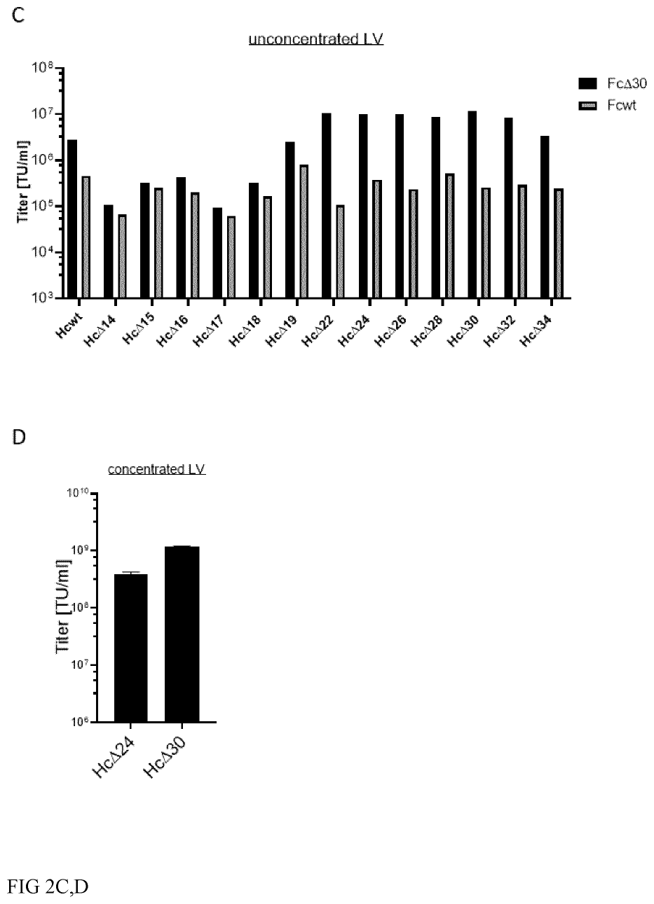

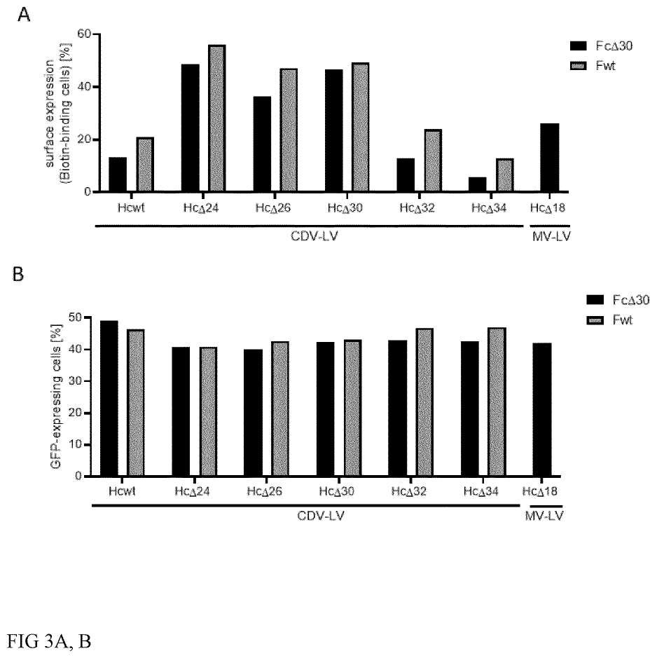

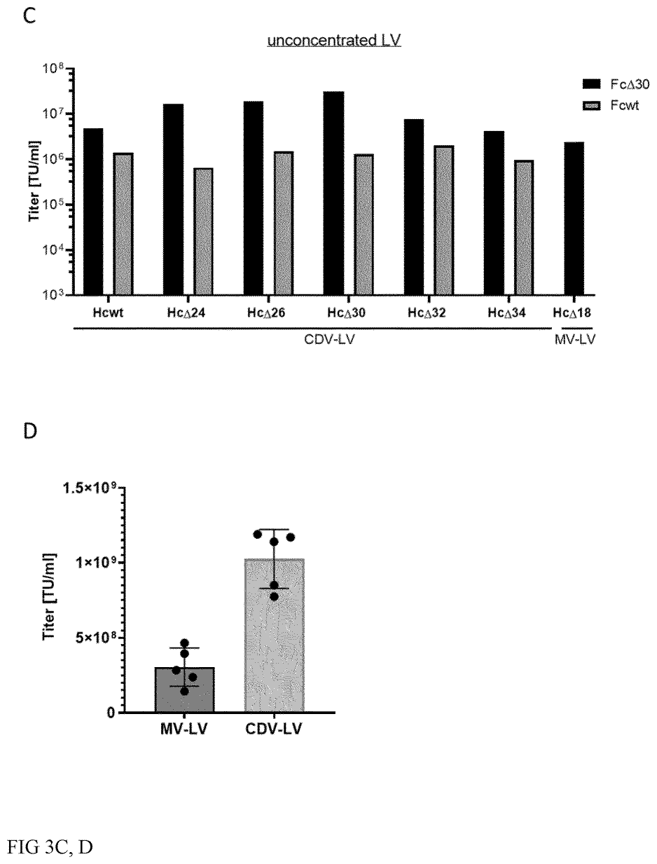

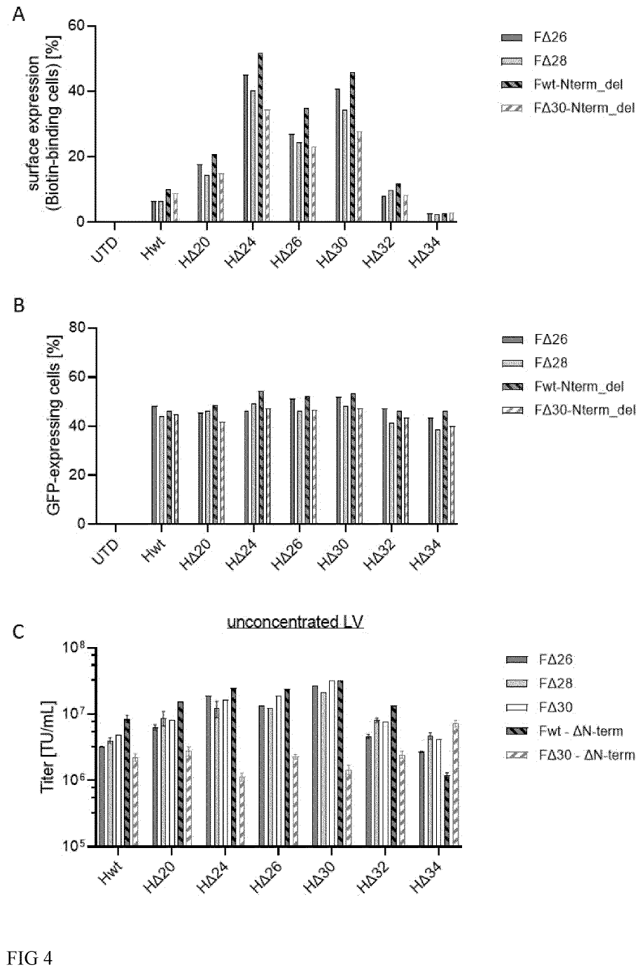

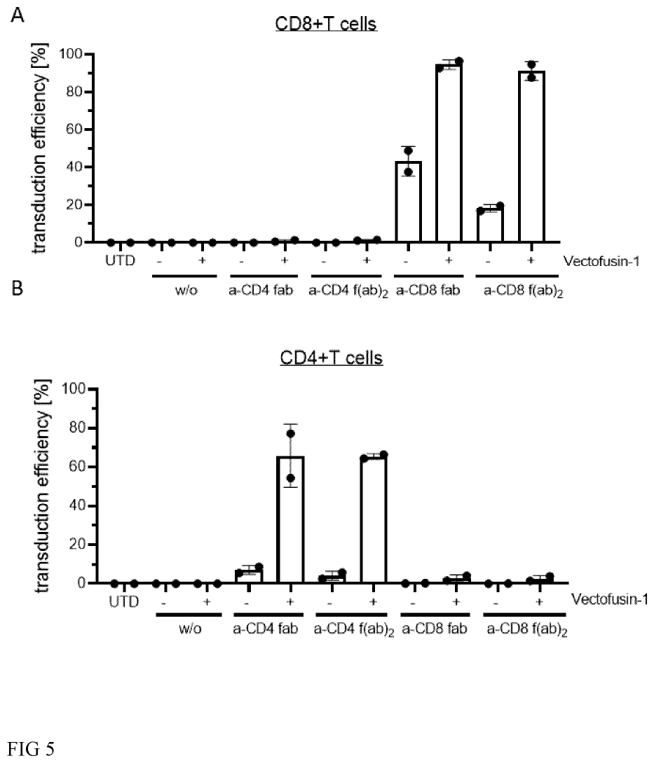

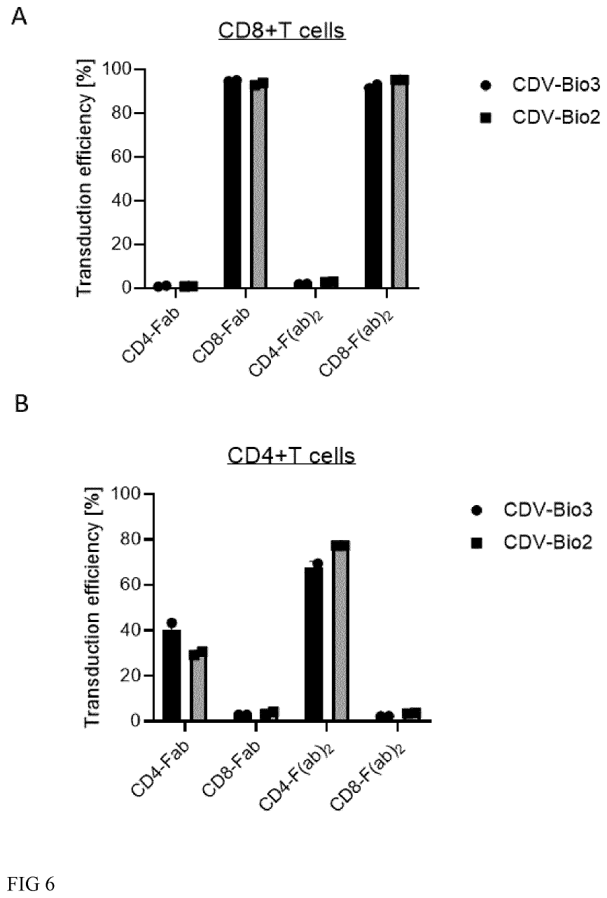

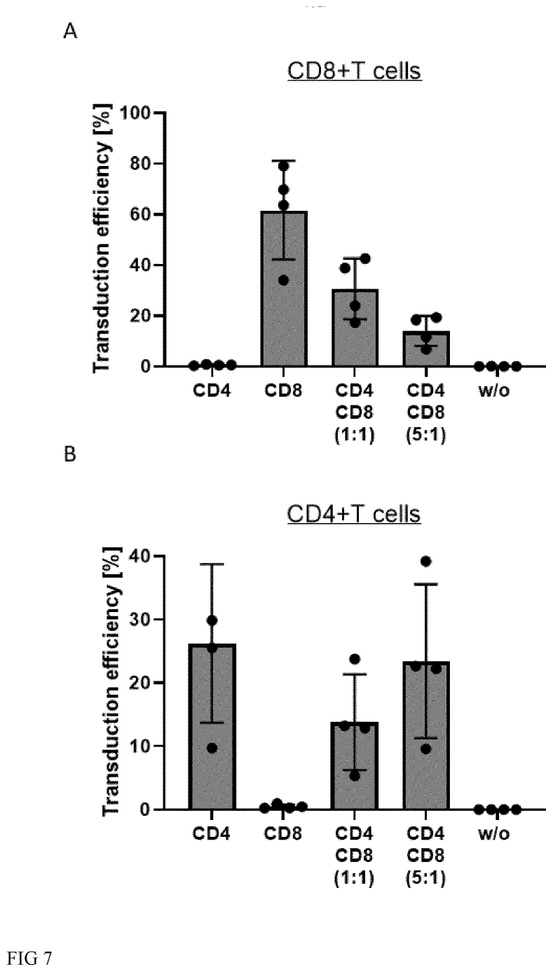

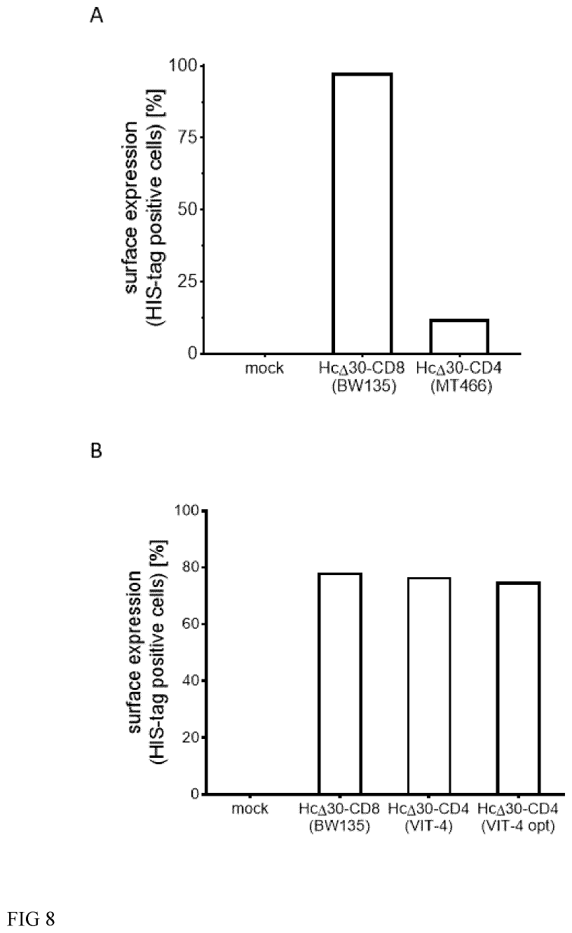

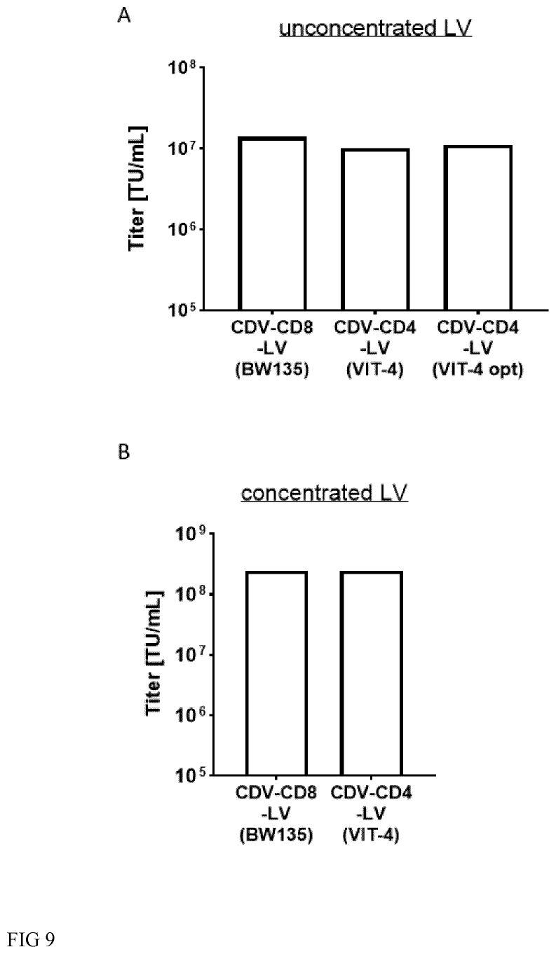

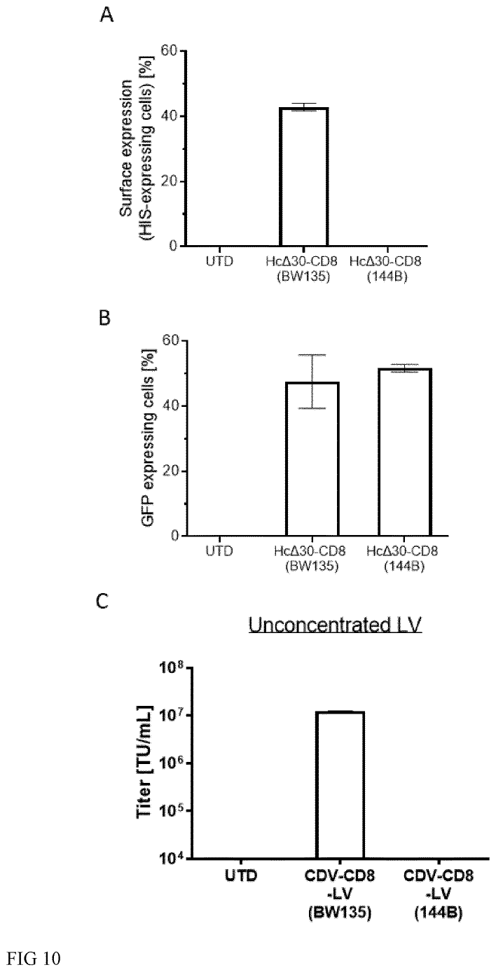

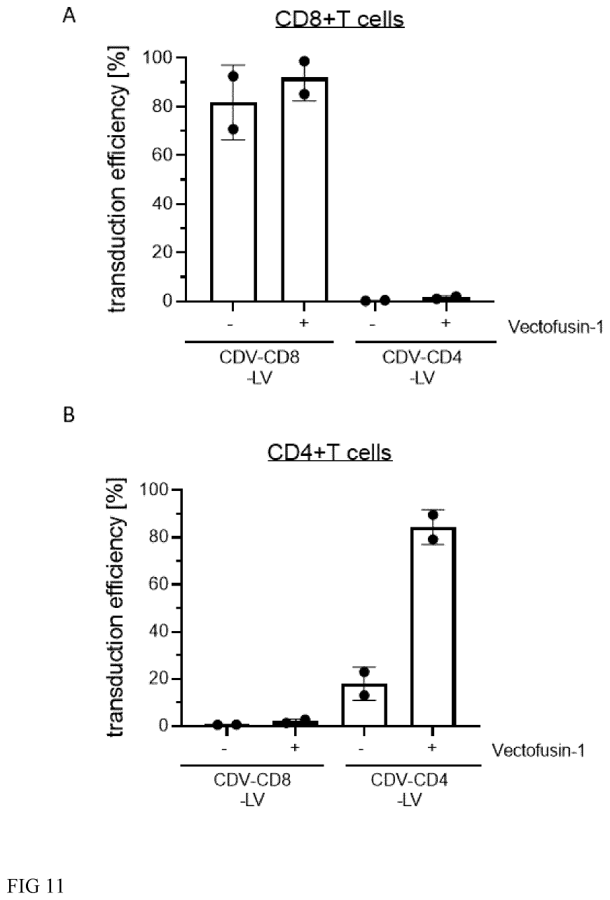

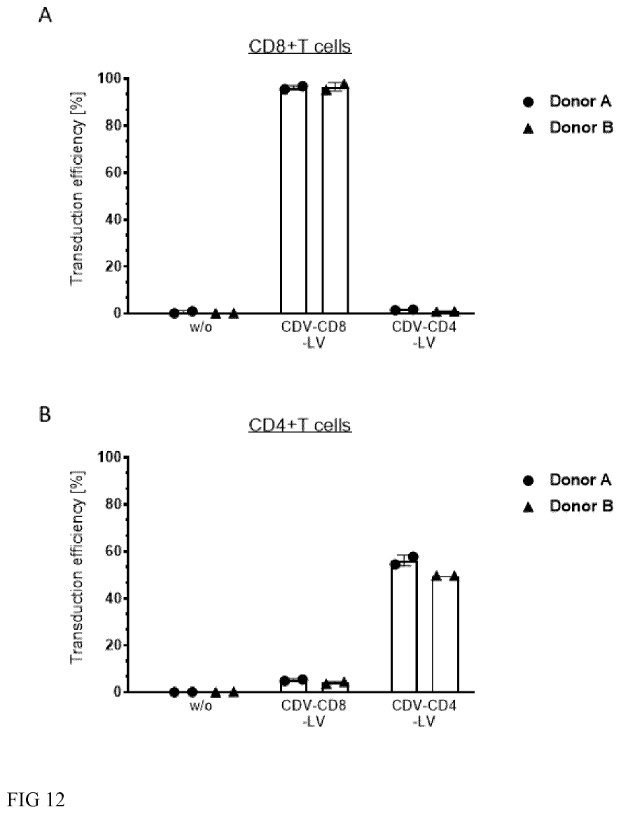

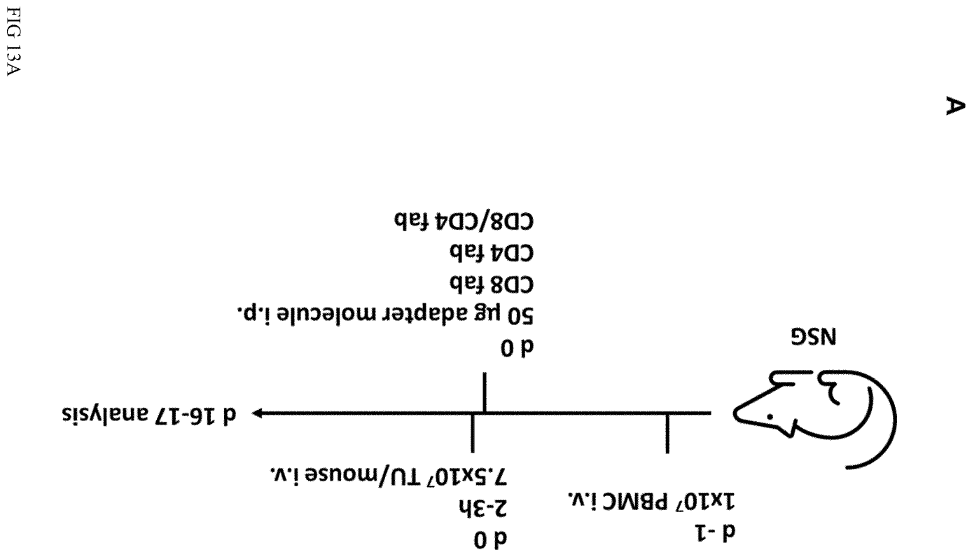

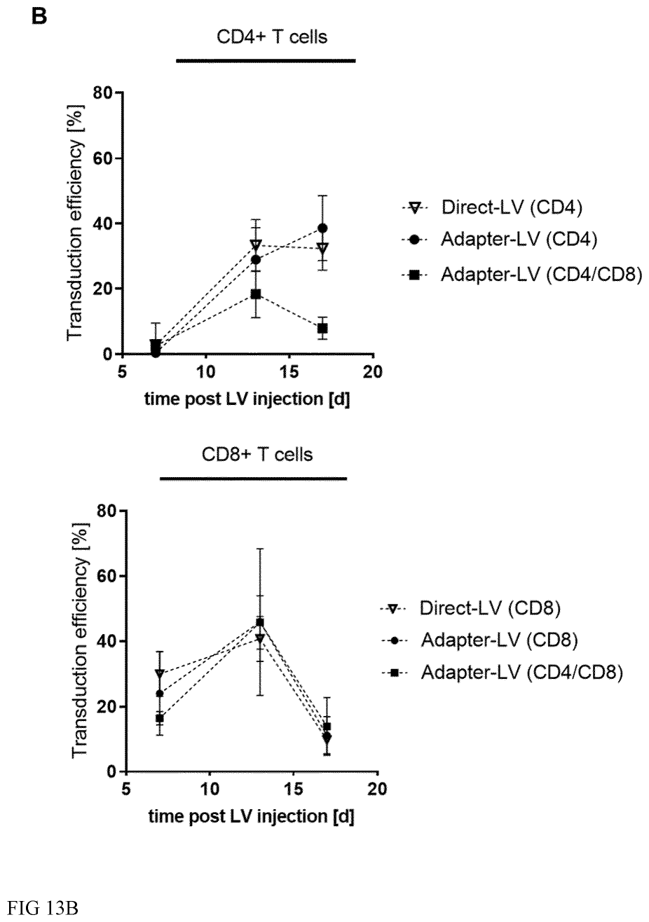

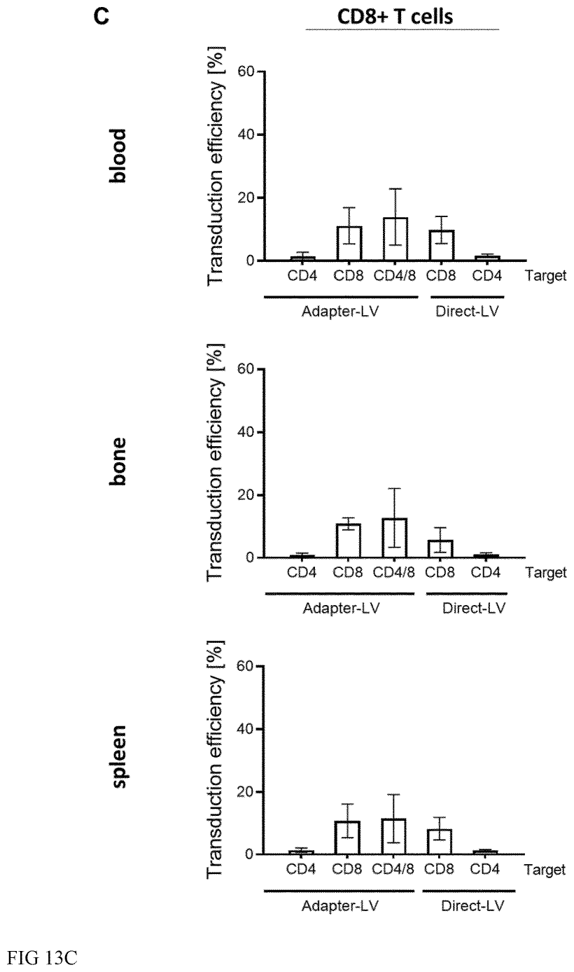

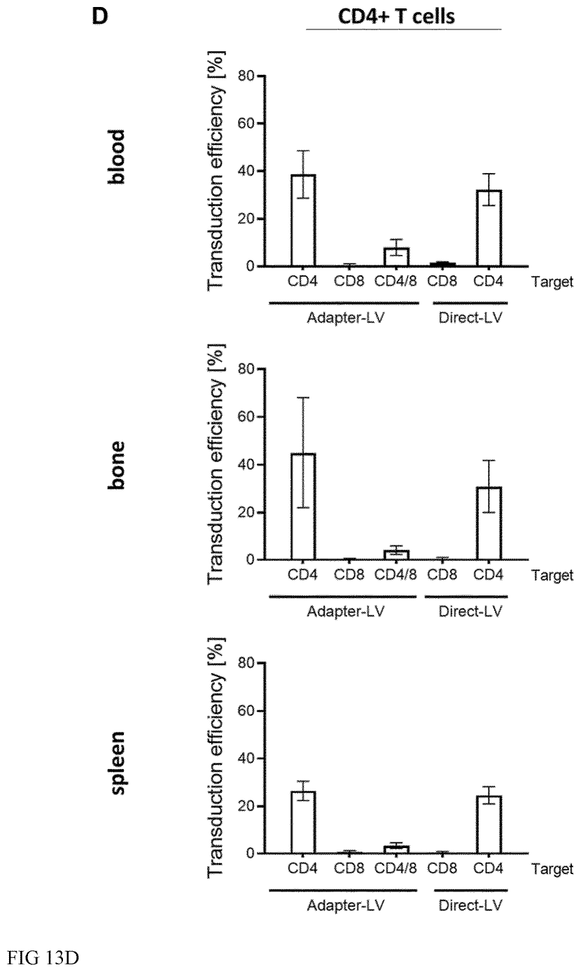

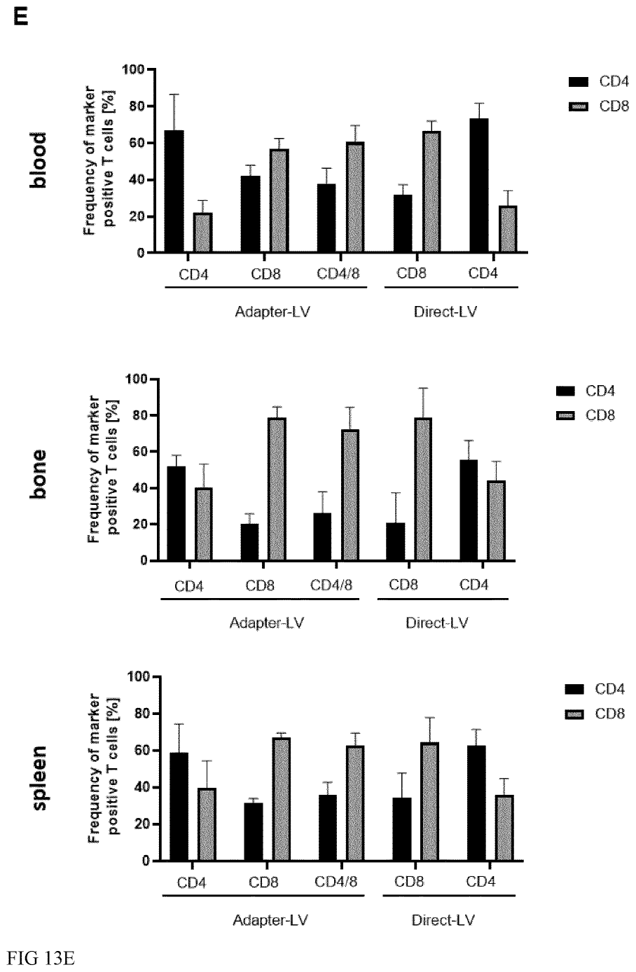

Retroviral vector particles can be pseudotyped with the H and F protein of canine distemper virus. Both envelope proteins are truncated in their cytoplasmic tail and the H protein is mutated to abolish binding to the natural receptors. To enable retargeting, the H protein is fused with a polypeptide specific for a target antigen (Direct-LV) or specific for a tag on an adapter molecule that mediates binding to the target antigen (Adapter-LV). While the Direct-LV can bind to cells expressing the target antigen, the Adapter-LV is only able to bind cells in presence of a tagged adapter. Two chains of the scFvs are linked via a 3(G4S) linker and may be present in either orientations (VH-VL or VL-VH). The orientation can influence expression levels, stability, affinity to the tag of the tagged polypeptide ant the titer of the pseudotyped retroviral vector or virus-like particle thereof, respectively. (FIG. 1)

### Example 2: Generation of CDV Pseudotyped Retroviral Vectors

Pseudotyped retroviral vector particles specific for a tag of tagged polypeptide or a specific target antigen were generated by transient transfection of HEK-293T cells. HEK-293T cells that were seeded in T175 flasks in DMEM/10% FCS (Biowest, Cat.No. 12362; Biochrom, Cat.No.S0415) the day before were transfected with a plasmid encoding for the CDV-H attachment protein, a plasmid encoding for the CDV-F fusion protein, a packaging plasmid encoding gag/pol/rev and a psi-positive transfer vector plasmid encoding GFP or CD20CAR. The pseudotyped retroviral vector particles were harvested 48 h post transfection. To remove cellular debris, the supernatant was collected, centrifuged for 10 min at 1000 rpm, followed by filtration through a 0.45 μm filter. To concentrate, the filtered supernatant was centrifuged through a 20% sucrose (Sigma Aldrich, Cat.No. 84097-250 g, 20% w/v in PBS) cushion for 24 h at 4° C. with 5350×g. The pelleted retroviral vectors were resuspended in 250 μl precooled PBS, aliquoted and stored at −80° C. for later use. To confirm sufficient transfection for production, the producer cells were stained after vector harvest for expression of the CDV H protein by staining with fluorescently labeled biotin. Subsequently, transfection efficiency was determined by quantification of the H protein and GFP expressing cells using flow cytometry (FIG. 2A,B; FIG. 3A,B; FIG. 4 A,B; FIG. 10A,B).

The transfection efficiency and expression of the transgenes is a prerequisite for efficient LV production. No significant differences in GFP expression confirmed successful transfection during the LV production (FIG. 2B, 3B, 4B, 10B). Surface expression of the H protein increased with increasing cytoplasmic tail truncation and peaked between 22 and 30 amino acid deletions (FIG. 2B). Surprisingly, the surface expression of the CDV H protein was increased compared to the measles H protein (FIG. 3A). Surface expression of the H proteins was independent of the co-expression with F proteins with different cytoplasmic tail truncations (FIG. 4A). Interestingly, surface expression of the H protein decreased upon co-expression with the F protein containing an cytoplasmic in combination with an N-terminal truncation (FIG. 4A). Surface expression of the H protein is strongly influenced by the sequence of the scFV. While H proteins fused to the biotin-specific scFV (SEQ ID NO: 18) and the CD8-specific scFV (SEQ ID NO: 9) were successfully expressed in HEK293T cells, no expression was achieved with H proteins fused to scFVs of SEQ ID 25 (FIG. 10A).

### Example 3: Titration of CDV Pseudotyped Retroviral Vectors

Pseudotyped retroviral vector particles were titrated on SupT1 cells. Adapter-LVs were titrated in the absence of the tagged polypeptide. Therefore, cell surface proteins were randomly labeled with LC-LC-biotin using by Biotin-LC-LC-NHS. SupT1 resuspended in 1 ml PBS were supplemented with Biotin-LC-LC-NHS followed by an incubation at 4° C. at constant mixing. After removing cell-free supernatant, cells were washed and seeded. For transduction SupT1 or biotinylated SupT1 were seeded with 2×10⁵ cells/well in 96-well in media (RPMI) without FCS. The LV particles were serially diluted in a RPMI and added to the cells or biotinylated cells. 96 h post transduction the transduction efficiency was determined by flow cytometry quantifying the ratio of GFP positive events. The ratio of GFP positive cells, the dilution factor and the volume of retroviral applied is used to calculate the retroviral vector titer (i.e. transducing units per volume (TU/ml) (FIG. 2 C-D, FIG. 3 C-D, FIG. 4 C, FIG. 9 A-B, FIG. 10C). The productivity of the CDV-LV increased with increasing truncation of the cytoplasmic tail of the H protein and peaked between a cytoplasmic tail truncation of 22-32 amino acids (FIG. 2C). Surprisingly, functional titers of CDV-LV were 10-fold higher compared to the MV-LV (FIG. 3C). Importantly, the highest productivity was achieved with a cytoplasmic tail truncation of both, the H and the F protein (FIG. 2C). The productivity peaked upon using HcΔ30 in combination with FcΔ26-30 (FIG. 4C). Interestingly, simultaneous truncation of the N-terminal signal sequence and the cytoplasmic tail of the F protein strongly decreased retroviral vector titers (FIG. 4 C).

### Example 4: Cell Surface Expression of the H Protein

Cell surface expression of the H proteins is crucial for successful incorporation into retroviral vector particles. Surface expression was determined by transient transfection of HEK293T cells. For that, HEK293T cells were seeded in 6 wells with a density of 8×10⁵ cells/well one day before transfection. The HEK293T cells were transfected with the plasmids encoding the H protein. Two days post transfection the cells were stained for expression of the H protein via a HIS tag using the respective antibody (Miltenyi Biotec), followed by flow cytometry to determine the ratio of His positive cells (FIG. 8 A,B). While CDV-H proteins fused to the scFV SEQ ID NO:6 and 9 were well expressed, only low expression levels were achieved for H proteins fused to the scFV with SEQ ID 24.

### Examples 5: Transduction Human T Cells in PBMC with Adapter-LV and Direct-LVs

Peripheral blood mononuclear cells of healthy donors were isolated from buffy coat using density gradient centrifugation. PBMC were seeded with 2.5×10⁵ cells/well in TexMACS™ medium supplemented with 12.5 ng/ml IL7 and 12.5 ng/ml IL15 and activated with TransAct™ overnight in a 96-well plate. If required, the cells were incubated with adapter molecules for 30 min at 4° C. prior to transduction. The cells were transduced with GFP or CD20CAR encoding Adapter-LVs or direct-targeted LV in presence or absence of Vectofusin-1® at a dose of 0.5-2.5 TU/cell. Vectofusin-1® was used according to the manufacturer's instructions. The cell culture medium was replaced with fresh medium two days later, followed by regular splitting of the cells in a ratio of 1:2 every other day. Transduction efficiency of CD8+ and CD4+ T cells was determined by flow cytometry five or twelve days post transduction after staining for the targeted antigens and the transduction marker (FIG. 5 A-B, FIG. 6 A-B, FIG. 7 A-B, FIG. 11 A-B, FIG. 12 A-B). Selective transduction of CD4+ and CD8+ T cells with all tested CDV-LVs was achieved.

### Examples 6: In Vivo Transduction with CDV-LVs

PBMC of a healthy donor were isolated from leukaphereses by density gradient centrifugation. After overnight incubation of PBMC in TexMACS™ medium (12.5 ng/ml IL-7, 12.5 ng/ml IL-15), 1×10⁷ viable CD45+ cells were injected i.v. into NSG® mice (Charles River, France). One day later, respective animals were i.p. injected with either 50 μg CD8-fab or CD4-fab adapter or with a mixture of both adapter molecules (25 μg CD4-fab+25 μg CD8-fab). Between 2-3 h post adapter injection LV was injected i.v. at a dose of 7.5×10⁷ TU/mouse. Mice were monitored regularly for their health status and weekly blood collection was performed to analyze the frequency of CAR-expressing T cells via flow cytometry. Mice were sacrificed 16-17 days post LV injection and blood, bone marrow and spleen samples were analyzed for frequency of CAR-expressing T cells by flow cytometry. The experimental workflow is shown in FIG. 13A. CAR-expressing CD4+ and CD8+ T cells were observed in blood as early as 7 d post transduction (FIG. 13B). Absence of CAR-expressing CD4+ T cells upon transduction with an CD8-mediated Adapter-LV or a CD8-specific Direct-LV and vice versa confirms selective transduction of the T cells in vivo (FIG. 13 B-D). In addition, the CD4/CD8 ratio of the T cells shifted towards the transduced subset, suggesting a survival or proliferative advantage of transduced cells (FIG. 13E).

Sequence listing

SEQ ID NO: 1 (OKT3 mut):
QVQLVQSGGGVVQPGRSLRLSCKASGYTFTRYTMHWVRQAPGKGLEWIGYINPSRG
YTNYNQKVKDRFTISRDNSKNTAFLQMDSLRPEDTGVYFCARYYDDHYSLDYWGQ
GTPVTVSSGGGGSGGGGSGGGGSDIQMTQSPSSLSASVGDRVTITCSASSSVSYMNW
YQQTPGKAPKRWIYDTSKLASGVPSRFSGSGSGTDYTFTISSLQPEDIATYYCQQWSS
NPFTFGQGTKLQIT
SEQ ID NO: 2 (env incorporation signal (gp41)):
NRVRQGYS
SEQ ID NO: 3 (CD8 transmembrane domain):
IYIWAPLAGTCGVLLLSL VITLYCKRGRKK
SEQ ID NO: 4 (CD4 scFV VL):
DIVMTQTPLSLPVSLGDQASISCRSSQILVDSNENTYLHWYLQKPGQSPKLLIYKVSNR
VSGVPDRFSGSGSGTDFTLKISRVEAEDLGVYFCSQNTHVPPTFGGGTKLEIK
SEQ ID NO: 5 (CD4 scFV VH):
EVQLQQSGPELVKPGASVKMSCKASGYTFTNYVLHWVKQNSGQGLEWIGFIDPYND
GAQYNEKFKGKATLTSDKSSSTVYMEFSSLTSEDSAVYYCTREGGIGGFAYWGQGTL
VTVSA
SEQ ID NO: 6 (CD4 scFV complete; clone Vit-4):
DIVMTQTPLSLPVSLGDQASISCRSSQILVDSNENTYLHWYLQKPGQSPKLLIYKVSNR
VSGVPDRFSGSGSGTDFTLKISRVEAEDLGVYFCSQNTHVPPTFGGGTKLEIKGGGGS
GGGGSGGGGSEVQLQQSGPELVKPGASVKMSCKASGYTFTNYVLHWVKQNSGQGL
EWIGFIDPYNDGAQYNEKFKGKATLTSDKSSSTVYMEFSSLTSEDSAVYYCTREGGIG
GFAYWGQGTLVTVSA
SEQ ID NO: 7 (CD8 scFV VL):
DIVLTQSPPSLAVSLGQRATISCRASKSVSTSGYRFLHWYQQKPGQAPKLLIYLASNLE
SGVPARFSGSGSGTDFTLNIHPVEEEDAATYYCQHSREFPWTFGGGTKLEIK
SEQ ID NO: 8 (CD8 scFV VH):
EVQLQQSGAEVVRSGASVKLTCTVSGFNIKDHYMHWVKQRPEQGLEWIGWIDPENG
DTEYDPKFQGKATMTADISSNTAYLQLNSLTSEETAVYYCNALDSSGYGFAYWGQG
TLVTVSA
SEQ ID NO: 9 (CD8 scFV complete, clone BW135):
DIVLTQSPPSLAVSLGQRATISCRASKSVSTSGYRFLHWYQQKPGQAPKLLIYLASNLE
SGVPARFSGSGSGTDFTLNIHPVEEEDAATYYCQHSREFPWTFGGGTKLEIKGGGGSG
GGGSGGGGSEVQLQQSGAEVVRSGASVKLTCTVSGFNIKDHYMHWVKQRPEQGLE
WIGWIDPENGDTEYDPKFQGKATMTADISSNTAYLQLNSLTSEETAVYYCNALDSSG
YGFAYWGQGTLVTVSA
SEQ ID NO: 10 (CDV H 5840P):
MLSYQDKVGAFYKDNARANSSKLSLVTEEQGGRRPPYLLFVLLILLVGIMTLLAITGV
RFHQVSTSNMEFSRLLKEDMEKSEAVHHQVIDVLTPLFKIIGDEVGLQLPQKLNEIKQ
FILQKTNFFNPNREFDFRDLHWCINPPSKIKVNFTNYCDTIGIKKSIASAANPILLSALS
GGRGDIFPPYRCSGATTSVGRVFPLSVSLSMSLISKTSEITSMLTAISDGVYGKTYLLVP
DYIEGEFDTQKIRVFEIGFIKRWLNNMPLLQTTNYMVLPENSKAKVCTIAVGELTLAS
LCVDESTVLLYHDSNGSQDGILVVTLGIFGATPMDQVEEVIPVAHPSVEKIHITNHRGF
IKDSIATWMVPALVSEKQEEQKNCLESACQRKTYPMCNQTSWEPFGGGQLPSYGRLT
LPLDPSIDLQLNISFTYGPVILNGDGMDYYESPLSDSGWLTIPPRNGTVLGLINKASRG
DQFTVIPHVLTFAPRESSGNCYLPIQTSQIMDKDVLTESNLVVLPTQNFRYVIATYDIS
RGDHAIVYYVYDPIRTISYTHPFRLTTKGRPDFLRIECFVWDDDLWCHQFYRFEADIT
NSTTSVENLVRIRFSCNRSKP
SEQ ID NO: 11 (CDV F 5840P):
MHNKIPKGSKTPTPTQQDPPPQHSIKSAETKTSQARYNITSAQQSTRHGPRTSDRPVH
YIMNRTRSCKQASYRSDNIPAHGDHEGIIHHTPESVSQGVRSRFKRRQSNATSSGSQC
TWLVLWCIGVASLFLCSKAQIHWNNLSTIGIIGTDSVHYKIMTRPSHQYLVIKLMPNV
SLIDNCTKAELGEYEKLLSSVLEPINQSLTLMTKNVKPLQSVGSGRRQRRFAGVVLAG
AALGVATAAQITAGIALHQSNLNAQAIQSLRTSLEQSNKAIEEIREATQETIIAVQGVQ
DYVNNELVPAMQHMSCELVGQRLGLKLLRYYTELLSIFGPSLRDPISAEISIQALSYAL
GGEIHKILEKLGYSGNDMIAILESRGIKTKITHVDIPGKLIILSISYPTLSEVKGVIVHRLE
AVSYNIGSQEWYTTVPRYVATNGYLISNFDESSCVFVSESAICSQNSLYPMSPLLQQCI
RGDTSSCARTLVSGTMGNKFILSKGNIVANCASILCKCYSTSTIINQSPDKLLTFIASDT
CPLVEIDGVTIQVGGRQYPDMVYESKVALGPAISLERLDVGTNLGNALKKLDDAKILI
DSSNQILETVRRSSFNFGSLLSVPILICTALALLFLIYCCKRRYQQTLKQHIKVDPTFKP
DLTGTSKSYVRSL
SEQ ID NO: 12 (N-terminal signal Sequence F protein):
MHNKIPKGSKTPTPTQQDPPPQHSIKSAETKTSQARYNITSAQQSTRHGPRTSDRPVH
YIMNRTRSCKQASYRSDNIPAHGDHEGIIHHTPESVSQGVRSRFKRRQSNATSSGSQC
TWLVLWCIGVASLFLCSKA
SEQ ID NO: 13 (cytoplasmic domain Fc430 of Canine distemper virus strain 5840P):
ALLFLIYCCKRR
SEQ ID NO: 14 (cytoplasmic domain of Hc421 of Canine distemper virus strain 5840P):
MKLSLVTEEQGGRRPPY
SEQ ID NO: 15 (cytoplasmic domain of HcA30 of Canine distemper virus strain 5840P):
MGRRPPY
SEQ ID NO: 16 (Bio3 18E3 Opt VH):
EVQLVESGGGLVKPGGSLKLSCAVSGFTFSNYAMSWVRQTPETRLEWVATITGGGS
YTYYPDSVKGRFTISRDNAKDTLYLQMSSLRSEDTAMYYCVRQRVGDYVSSLLGYW
GQGTLVTVSA
SEQ ID NO: 17 (Bio3 18E3 Opt VL):
DVVMTQTPLSLPVSLGDQASISCRSSQSLVHSNGNTYLHWFLQKPGQSPNLLIYKVSN
RFSGVPDRFSGSGSGTDFTLKISRVEAEDLGVYFCSQSTHVPYTFGGGTKLEIK
SEQ ID NO: 18 (Bio3 18E3 Opt complete):
DVVMTQTPLSLPVSLGDQASISCRSSQSLVHSNGNTYLHWFLQKPGQSPNLLIYKVSN
RFSGVPDRFSGSGSGTDFTLKISRVEAEDLGVYFCSQSTHVPYTFGGGTKLEIKGGGG
SGGGGSGGGGSEVQLVESGGGLVKPGGSLKLSCAVSGFTFSNYAMSWVRQTPETRL
EWVATITGGGSYTYYPDSVKGRFTISRDNAKDTLYLQMSSLRSEDTAMYYCVRQRV
GDYVSSLLGYWGQGTLVTVSA
SEQ ID NO: 19 (Bio2 4G10 Opt VH):
EVQLQQSGAELVKPGASVKLSCTASGFNSKDTYMQWVKQRPEQGLEWIGRIDPANG
NSKYDPKFQGKATITADTSSNTAYLQLSSLTSEDTAVYYCARWDYYGSTSAWFAYW
GQGTLVTVSA
SEQ ID NO: 20 (Bio2 4G10 Opt VL):
DIQMTQSPASLSASVGDTVTITCRASGNIHNYLTWYQQKPGKSPQLLVYNAKTLADG
VPSRFSGSGSGTQYSLKINSLQPEDLGSYYCQHFWSTPYTFGGGTKLEIK
SEQ ID NO: 21 (Bio2 4G10 Opt complete):
DIQMTQSPASLSASVGDTVTITCRASGNIHNYLTWYQQKPGKSPQLLVYNAKTLADG
VPSRFSGSGSGTQYSLKINSLQPEDLGSYYCQHFWSTPYTFGGGTKLEIKGGGGSGGG
GSGGGGSEVQLQQSGAELVKPGASVKLSCTASGFNSKDTYMQWVKQRPEQGLEWIG
RIDPANGNSKYDPKFQGKATITADTSSNTAYLQLSSLTSEDTAVYYCARWDYYGSTS
AWFAYWGQGTLVTVSA
SEQ ID NO: 22 (Fwt with N-terminal truncation)
MRTRSCKQASYRSDNIPAHGDHEGIIHHTPESVSQGVRSRFKRRQSNATSSGSQCTWL
VLWCIGVASLFLCSKAQIHWNNLSTIGIIGTDSVHYKIMTRPSHQYLVIKLMPNVSLID
NCTKAELGEYEKLLSSVLEPINQSLTLMTKNVKPLQSVGSGRRQRRFAGVVLAGAAL
GVATAAQITAGIALHQSNLNAQAIQSLRTSLEQSNKAIEEIREATQETIIAVQGVQDYV
NNELVPAMQHMSCELVGQRLGLKLLRYYTELLSIFGPSLRDPISAEISIQALSYALGGE
IHKILEKLGYSGNDMIAILESRGIKTKITHVDIPGKLIILSISYPTLSEVKGVIVHRLEAVS
YNIGSQEWYTTVPRYVATNGYLISNFDESSCVFVSESAICSQNSLYPMSPLLQQCIRGD
TSSCARTLVSGTMGNKFILSKGNIVANCASILCKCYSTSTIINQSPDKLLTFIASDTCPL
VEIDGVTIQVGGRQYPDMVYESKVALGPAISLERLDVGTNLGNALKKLDDAKILIDSS
NQILETVRRSSFNFGSLLSVPILICTALALLFLIYCCKRRYQQTLKQHIKVDPTFKPDLT
GTSKSYVRSL
SEQ ID NO: 23 (FcD30 with N-terminal truncation)
MRTRSCKQASYRSDNIPAHGDHEGIIHHTPESVSQGVRSRFKRRQSNATSSGSQCTWL
VLWCIGVASLFLCSKAQIHWNNLSTIGIIGTDSVHYKIMTRPSHQYLVIKLMPNVSLID
NCTKAELGEYEKLLSSVLEPINQSLTLMTKNVKPLQSVGSGRRQRRFAGVVLAGAAL
GVATAAQITAGIALHQSNLNAQAIQSLRTSLEQSNKAIEEIREATQETIIAVQGVQDYV
NNELVPAMQHMSCELVGQRLGLKLLRYYTELLSIFGPSLRDPISAEISIQALSYALGGE
IHKILEKLGYSGNDMIAILESRGIKTKITHVDIPGKLIILSISYPTLSEVKGVIVHRLEAVS
YNIGSQEWYTTVPRYVATNGYLISNFDESSCVFVSESAICSQNSLYPMSPLLQQCIRGD
TSSCARTLVSGTMGNKFILSKGNIVANCASILCKCYSTSTIINQSPDKLLTFIASDTCPL
VEIDGVTIQVGGRQYPDMVYESKVALGPAISLERLDVGTNLGNALKKLDDAKILIDSS
NQILETVRRSSFNFGSLLSVPILICTALALLFLIYCCKRR
SEQ ID NO: 24 (CD4 scFV complete, clone MT466):
DIVLTQSPASLAVSLGQRATISCKASQSVDYDGDSYMNWYQQKPGQPPKLLIYAASN
LESGIPDRFSGSGSGTDFIFNIHPVEEEDAATYFCQQFNEDPPTFGGGTKLEIKGGGGS
GGGGSGGGGSQIQLQQSGPELVKPGASVKMSCKASGYTFTDYVIHWVKQRTGQGLE
WIGEIYPGSGSSYYNENLKGKATLTADKSSNTVYILLSSLTSEDSAVYFCARRKVQGF
AYWGQGTLVTVSA
SEQ ID NO: 25 (CD8 scFV complete; clone 144B):
DVQITQSPSYLAASPGETITINCRASKSISKYLAWYQEKPGKTKKFLIYFGSTLQSGIPS
RFSGSGSGTDFTLTISSLEPEDFAMYYCQQHDEYPLTFGAGTKLEIKGGGGSGGGGSG
GGGSQVQLQQSGPELVRPGVSVKISCKGSGYTFTDYAMHWVKQSHAKSLEWIGIIST
YYGNTKYNQKFKGKATMTVDKSSSTAYMELARLTSEDSAIYYCARKGDGNGAMDS
WGQGTSVTVSS

### REFERENCES

Anliker B, Abel T, Kneissl S, Hlavaty J, Caputi A, Brynza J, Schneider I C, Münch R C, Petznek H, Kontermann R E, Koehl U, Johnston I C, Keinanen K, Müller U C, Hohenadl C, Monyer H, Cichutek K, Buchholz C J. Specific gene transfer to neurons, endothelial cells and hematopoietic progenitors with lentiviral vectors. Nat Methods. 2010 November; 7(11):929-35. doi: 10.1038/nmeth.1514. Epub 2010 Oct. 10. PMID: 20935652.
Amirache F, Lévy C, Costa C, Mangeot P E, Torbett B E, Wang C X, Negre D, Cosset F L, Verhoeyen E. Mystery solved: VSV-G-LVs do not allow efficient gene transfer into unstimulated T cells, B cells, and HSCs because they lack the LDL receptor. Blood. 2014 Feb. 27; 123(9):1422-4. doi: 10.1182/blood-2013-11-540641. Erratum in: Blood. 2014 Jun. 5; 123(23):3682. PMID: 24578496.
Bah E S, Nace R A, Peng K W, Muñoz-Alia M Á, Russell S J. Retargeted and Stealth-Modified Oncolytic Measles Viruses for Systemic Cancer Therapy in Measles Immune Patients. Mol Cancer Ther. 2020 October; 19(10):2057-2067. doi: 10.1158/1535-7163.MCT-20-0134. Epub 2020 Aug. 26. PMID: 32847970; PMCID: PMC7875472.
Bender R R, Muth A, Schneider I C, Friedel T, Hartmann J, Plückthun A, Maisner A, Buchholz C J. Receptor-Targeted Nipah Virus Glycoproteins Improve Cell-Type Selective Gene Delivery and Reveal a Preference for Membrane-Proximal Cell Attachment. PLoS Pathog. 2016 Jun. 9; 12(6):e1005641. doi: 10.1371/journal.ppat.1005641. PMID: 27281338; PMCID: PMC4900575.
Kneissl S, Abel T, Rasbach A, Brynza J, Schneider-Schaulies J, Buchholz C J. Measles virus glycoprotein-based lentiviral targeting vectors that avoid neutralizing antibodies. PLoS One. 2012; 7(10):e46667. doi: 10.1371/journal.pone.0046667. Epub 2012 Oct. 10. PMID: 23071609; PMCID: PMC3468630.
Miest T S, Yaiw K C, Frenzke M, Lampe J, Hudacek A W, Springfeld C, von Messling V, Ungerechts G, Cattaneo R. Envelope-chimeric entry-targeted measles virus escapes neutralization and achieves oncolysis. Mol Ther. 2011 October; 19(10):1813-20. doi: 10.1038/mt.2011.92. Epub 2011 May 24. PMID: 21610701; PMCID: PMC3188758.
Muñoz-Alia M Á, Nace R A, Tischer A, Zhang L, Bah E S, Auton M, Russell S J. MeV-Stealth: A CD46-specific oncolytic measles virus resistant to neutralization by measles-immune human serum. PLoS Pathog. 2021 Feb. 3; 17(2):e1009283. doi: 10.1371/journal.ppat.1009283. PMID: 33534834; PMCID: PMC7886131.
Muñoz-Alia M Á & Russell S J. Stealthed, Retargeted HIV-1 Vectors Incorporating Darpin-Displaying Canine Distemper Virus Envelope Glycoproteins without Cytoplasmic Tail Truncations. Mol Ther. 2021 Apr. 27; 29

1-14. (canceled)

15. A pseudotyped retroviral vector particle for delivering a transgene into a target cell, wherein said retroviral vector particle comprises the following components:
a) an antigen binding protein that contains protein H of a canine distemper virus (CDV) truncated at its N-terminal and that does not interact with at least one of its native receptors, fused at its ectodomain to a polypeptide comprising an antigen binding domain that specifically binds to a target antigen expressed on the surface of the target cell; and
b) an envelope protein with fusion activity that contains protein F of CDV set forth in SEQ. ID NO:11, truncated at its C-terminus by deletion of at least Fc Δ26-Δ30;
wherein said retroviral vector particle is a lentiviral or gamma retroviral vector particle that contains at least one nucleic acid molecule encoding a transgene.

16. An improved retroviral vector particle delivering a transgene into a target cell,
wherein said retroviral vector particle is a pseudotyped lentiviral or gamma retroviral particle that comprises the following components:
a) an antigen binding protein that contains protein H of a Paramyxoviridae family virus that has been recombinantly modified so that does not interact with at least one of its native receptors, but instead binds to a target antigen expressed on the surface of said target cell, or to a tag on a tagged polypeptide that specifically binds said antigen;
b) an envelope protein with fusion activity; and
c) at least one nucleic acid molecule encoding a transgene;
wherein the improvement comprises using for component (b) a protein that contains protein F of CDV set forth in SEQ. ID NO:11, truncated at its C-terminus by deletion of at least Fc Δ26-Δ30.

17. The pseudotyped retroviral vector particle of claim 15, wherein component (b) comprises SEQ ID NO:12.

18. The pseudotyped retroviral vector particle of claim 15, wherein component (a) is Hc A21-Δ32 as compared with unmodified protein H set forth in SEQ ID NO: 10.

19. The pseudotyped retroviral vector particle of claim 15, wherein component (a) comprises amino acid substitutions D526A, I527S, S528A, R529A, Y547A and T548A as compared with unmodified protein H set forth in SEQ ID NO: 10.

20. The improved retroviral vector particle of claim 16, wherein component (a) is Hc A21-Δ32 as compared with unmodified protein H set forth in SEQ ID NO: 10.

21. The improved retroviral vector particle of claim 16, wherein component (a) comprises amino acid substitutions D526A, I527S, S528A, R529A, Y547A and T548A as compared with unmodified protein H set forth in SEQ ID NO: 10.

22. The pseudotyped retroviral vector particle of claim 15, wherein component (a) binds specifically to CD4 on a target cell.

23. The pseudotyped retroviral vector particle of claim 22, wherein component (a) comprises SEQ ID NO:4 (V_{L}) and SEQ ID NO: 5 (V_{H}).

24. The pseudotyped retroviral vector particle of claim 15, wherein component (a) binds specifically to CD8 on a target cell.

25. The pseudotyped retroviral vector particle of claim 24, wherein component (a) comprises SEQ ID NO:7 (V_{L}) and SEQ ID NO:8 (V_{H}).

26. The pseudotyped retroviral vector particle of claim 15, wherein component (a) binds specifically to a tag on said tagged polypeptide.

27. The pseudotyped retroviral vector particle of claim 26, wherein the tag on said tagged polypeptide is a hapten, dextran, or a polypeptide.

28. The pseudotyped retroviral vector particle of claim 26, in combination with said tagged polypeptide.

29. The combination of claim 28, wherein the tagged polypeptide binds specifically to CD4 on a target cell.

30. The combination of claim 29, wherein the tagged polypeptide comprises SEQ ID NO:4 (V_{L}) and SEQ ID NO: 5 (V_{H}).

31. The combination of claim 29, wherein the tagged polypeptide binds specifically to CD8 on a target cell.

32. The combination of claim 31, wherein the tagged polypeptide comprises SEQ ID NO:7 (V_{L}) and SEQ ID NO:8 (V_{H}).

33. The combination of 26, wherein the tag of said tagged polypeptide is biotin,
and wherein component (a) comprises either SEQ ID NO: 16 (V_{L}) and SEQ ID NO: 17 (V_{H}); or SEQ ID NO: 19 (V_{L}) and SEQ ID NO:20 (V_{H}).

34. A method of preparing a pseudotyped retroviral vector particle for delivering a transgene into a target cell, the method comprising:
(1) selecting a target antigen on the target cell;
(2) constructing a tagged polypeptide that binds specifically to the selected target antigen;
(3) designing an antigen binding protein that comprises a truncated protein H of a virus of the Paramyxoviridae family that does not interact with at least one of its native receptors, fused at its ectodomain to a polypeptide comprising an antigen binding domain that specifically binds to a tag on said tagged polypeptide;
(4) constructing a pseudotyped Paramyxoviridae viral vector that bears (a) said antigen binding protein and (b) a fusion protein that contains protein F of CDV set forth in SEQ. ID NO:11, truncated at its C-terminus by deletion of at least Fc Δ26-Δ30; and
(5) packaging said transgene in the pseudotyped viral vector;
wherein contacting the target cell with said viral vector in the presence of said tagged polypeptide results in delivery of said gene or protein into the target cell.

35. The method of claim 34, wherein the tag on said tagged polypeptide is a hapten, dextran or a peptide.

36. The method of claim 34, wherein said envelope protein with fusion activity comprises SEQ ID NO:12.

37. The method of claim 35, wherein said antigen binding protein is Hc A21-Δ32 as compared with unmodified protein H set forth in SEQ ID NO: 10.

38. The method of claim 35, wherein said antigen binding protein comprises amino acid substitutions D526A, I527S, S528A, R529A, Y547A and T548A compared with unmodified protein H set forth in SEQ ID NO: 10.


SEQUENCE LISTING (25 sequences)

SEQ ID NO: 1 - protein, 240 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 2 - protein, 8 aa, synthetic construct
NRVRQGYS

SEQ ID NO: 3 - protein, 30 aa, synthetic construct
IYIWAPLAGTCGVLLLSLVITLYCKRGRKK

SEQ ID NO: 4 - protein, 112 aa, synthetic construct
DIVMTQTPLSLPVSLGDQASISCRSSQILVDSNENTYLHWYLQKPGQSPKLLIYKVSNRVSGVPDRFSGSGSGTDFTLKISRVEAEDLGVYFCSQNTHVPPTFGGGTKLEIK

SEQ ID NO: 5 - protein, 118 aa, synthetic construct
EVQLQQSGPELVKPGASVKMSCKASGYTFTNYVLHWVKQNSGQGLEWIGFIDPYNDGAQYNEKFKGKATLTSDKSSSTVYMEFSSLTSEDSAVYYCTREGGIGGFAYWGQGTLVTVSA

SEQ ID NO: 6 - protein, 245 aa, synthetic construct
DIVMTQTPLSLPVSLGDQASISCRSSQILVDSNENTYLHWYLQKPGQSPKLLIYKVSNRVSGVPDRFSGSGSGTDFTLKISRVEAEDLGVYFCSQNTHVPPTFGGGTKLEIKGGGGSGGGGSGGGGSEVQLQQSGPELVKPGASVKMSCKASGYTFTNYVLHWVKQNSGQGLEWIGFIDPYNDGAQYNEKFKGKATLTSDKSSSTVYMEFSSLTSEDSAVYYCTREGGIGGFAYWGQGTLVTVSA

SEQ ID NO: 7 - protein, 111 aa, synthetic construct
DIVLTQSPPSLAVSLGQRATISCRASKSVSTSGYRFLHWYQQKPGQAPKLLIYLASNLESGVPARFSGSGSGTDFTLNIHPVEEEDAATYYCQHSREFPWTFGGGTKLEIK

SEQ ID NO: 8 - protein, 119 aa, synthetic construct
EVQLQQSGAEVVRSGASVKLTCTVSGFNIKDHYMHWVKQRPEQGLEWIGWIDPENGDTEYDPKFQGKATMTADISSNTAYLQLNSLTSEETAVYYCNALDSSGYGFAYWGQGTLVTVSA

SEQ ID NO: 9 - protein, 245 aa, synthetic construct
DIVLTQSPPSLAVSLGQRATISCRASKSVSTSGYRFLHWYQQKPGQAPKLLIYLASNLESGVPARFSGSGSGTDFTLNIHPVEEEDAATYYCQHSREFPWTFGGGTKLEIKGGGGSGGGGSGGGGSEVQLQQSGAEVVRSGASVKLTCTVSGFNIKDHYMHWVKQRPEQGLEWIGWIDPENGDTEYDPKFQGKATMTADISSNTAYLQLNSLTSEETAVYYCNALDSSGYGFAYWGQGTLVTVSA

SEQ ID NO: 10 - protein, 607 aa, synthetic construct
MLSYQDKVGAFYKDNARANSSKLSLVTEEQGGRRPPYLLFVLLILLVGIMTLLAITGVRFHQVSTSNMEFSRLLKEDMEKSEAVHHQVIDVLTPLFKIIGDEVGLQLPQKLNEIKQFILQKTNFFNPNREFDFRDLHWCINPPSKIKVNFTNYCDTIGIKKSIASAANPILLSALSGGRGDIFPPYRCSGATTSVGRVFPLSVSLSMSLISKTSEITSMLTAISDGVYGKTYLLVPDYIEGEFDTQKIRVFEIGFIKRWLNNMPLLQTTNYMVLPENSKAKVCTIAVGELTLASLCVDESTVLLYHDSNGSQDGILVVTLGIFGATPMDQVEEVIPVAHPSVEKIHITNHRGFIKDSIATWMVPALVSEKQEEQKNCLESACQRKTYPMCNQTSWEPFGGGQLPSYGRLTLPLDPSIDLQLNISFTYGPVILNGDGMDYYESPLSDSGWLTIPPRNGTVLGLINKASRGDQFTVIPHVLTFAPRESSGNCYLPIQTSQIMDKDVLTESNLVVLPTQNFRYVIATYDISRGDHAIVYYVYDPIRTISYTHPFRLTTKGRPDFLRIECFVWDDDLWCHQFYRFEADITNSTTSVENLVRIRFSCNRSKP

SEQ ID NO: 11 - protein, 662 aa, synthetic construct
MHNKIPKGSKTPTPTQQDPPPQHSIKSAETKTSQARYNITSAQQSTRHGPRTSDRPVHYIMNRTRSCKQASYRSDNIPAHGDHEGIIHHTPESVSQGVRSRFKRRQSNATSSGSQCTWLVLWCIGVASLFLCSKAQIHWNNLSTIGIIGTDSVHYKIMTRPSHQYLVIKLMPNVSLIDNCTKAELGEYEKLLSSVLEPINQSLTLMTKNVKPLQSVGSGRRQRRFAGVVLAGAALGVATAAQITAGIALHQSNLNAQAIQSLRTSLEQSNKAIEEIREATQETIIAVQGVQDYVNNELVPAMQHMSCELVGQRLGLKLLRYYTELLSIFGPSLRDPISAEISIQALSYALGGEIHKILEKLGYSGNDMIAILESRGIKTKITHVDIPGKLIILSISYPTLSEVKGVIVHRLEAVSYNIGSQEWYTTVPRYVATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGVTIQVGGRQYPDMVYESKVALGPAISLERLDVGTNLGNALKKLDDAKILIDSSNQILETVRRSSFNFGSLLSVPILICTALALLFLIYCCKRRYQQTLKQHIKVDPTFKPDLTGTSKSYVRSL

SEQ ID NO: 12 - protein, 135 aa, synthetic construct
MHNKIPKGSKTPTPTQQDPPPQHSIKSAETKTSQARYNITSAQQSTRHGPRTSDRPVHYIMNRTRSCKQASYRSDNIPAHGDHEGIIHHTPESVSQGVRSRFKRRQSNATSSGSQCTWLVLWCIGVASLFLCSKA

SEQ ID NO: 13 - protein, 12 aa, synthetic construct
ALLFLIYCCKRR

SEQ ID NO: 14 - protein, 17 aa, synthetic construct
MKLSLVTEEQGGRRPPY

SEQ ID NO: 15 - protein, 7 aa, synthetic construct
MGRRPPY

SEQ ID NO: 16 - protein, 122 aa, synthetic construct
EVQLVESGGGLVKPGGSLKLSCAVSGFTFSNYAMSWVRQTPETRLEWVATITGGGSYTYYPDSVKGRFTISRDNAKDTLYLQMSSLRSEDTAMYYCVRQRVGDYVSSLLGYWGQGTLVTVSA

SEQ ID NO: 17 - protein, 112 aa, synthetic construct
DVVMTQTPLSLPVSLGDQASISCRSSQSLVHSNGNTYLHWFLQKPGQSPNLLIYKVSNRFSGVPDRFSGSGSGTDFTLKISRVEAEDLGVYFCSQSTHVPYTFGGGTKLEIK

SEQ ID NO: 18 - protein, 249 aa, synthetic construct
DVVMTQTPLSLPVSLGDQASISCRSSQSLVHSNGNTYLHWFLQKPGQSPNLLIYKVSNRFSGVPDRFSGSGSGTDFTLKISRVEAEDLGVYFCSQSTHVPYTFGGGTKLEIKGGGGSGGGGSGGGGSEVQLVESGGGLVKPGGSLKLSCAVSGFTFSNYAMSWVRQTPETRLEWVATITGGGSYTYYPDSVKGRFTISRDNAKDTLYLQMSSLRSEDTAMYYCVRQRVGDYVSSLLGYWGQGTLVTVSA

SEQ ID NO: 19 - protein, 122 aa, synthetic construct
EVQLQQSGAELVKPGASVKLSCTASGFNSKDTYMQWVKQRPEQGLEWIGRIDPANGNSKYDPKFQGKATITADTSSNTAYLQLSSLTSEDTAVYYCARWDYYGSTSAWFAYWGQGTLVTVSA

SEQ ID NO: 20 - protein, 107 aa, synthetic construct
DIQMTQSPASLSASVGDTVTITCRASGNIHNYLTWYQQKPGKSPQLLVYNAKTLADGVPSRFSGSGSGTQYSLKINSLQPEDLGSYYCQHFWSTPYTFGGGTKLEIK

SEQ ID NO: 21 - protein, 244 aa, synthetic construct
DIQMTQSPASLSASVGDTVTITCRASGNIHNYLTWYQQKPGKSPQLLVYNAKTLADGVPSRFSGSGSGTQYSLKINSLQPEDLGSYYCQHFWSTPYTFGGGTKLEIKGGGGSGGGGSGGGGSEVQLQQSGAELVKPGASVKLSCTASGFNSKDTYMQWVKQRPEQGLEWIGRIDPANGNSKYDPKFQGKATITADTSSNTAYLQLSSLTSEDTAVYYCARWDYYGSTSAWFAYWGQGTLVTVSA

SEQ ID NO: 22 - protein, 601 aa, synthetic construct
MRTRSCKQASYRSDNIPAHGDHEGIIHHTPESVSQGVRSRFKRRQSNATSSGSQCTWLVLWCIGVASLFLCSKAQIHWNNLSTIGIIGTDSVHYKIMTRPSHQYLVIKLMPNVSLIDNCTKAELGEYEKLLSSVLEPINQSLTLMTKNVKPLQSVGSGRRQRRFAGVVLAGAALGVATAAQITAGIALHQSNLNAQAIQSLRTSLEQSNKAIEEIREATQETIIAVQGVQDYVNNELVPAMQHMSCELVGQRLGLKLLRYYTELLSIFGPSLRDPISAEISIQALSYALGGEIHKILEKLGYSGNDMIAILESRGIKTKITHVDIPGKLIILSISYPTLSEVKGVIVHRLEAVSYNIGSQEWYTTVPRYVATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGVTIQVGGRQYPDMVYESKVALGPAISLERLDVGTNLGNALKKLDDAKILIDSSNQILETVRRSSFNFGSLLSVPILICTALALLFLIYCCKRRYQQTLKQHIKVDPTFKPDLTGTSKSYVRSL

SEQ ID NO: 23 - protein, 571 aa, synthetic construct
MRTRSCKQASYRSDNIPAHGDHEGIIHHTPESVSQGVRSRFKRRQSNATSSGSQCTWLVLWCIGVASLFLCSKAQIHWNNLSTIGIIGTDSVHYKIMTRPSHQYLVIKLMPNVSLIDNCTKAELGEYEKLLSSVLEPINQSLTLMTKNVKPLQSVGSGRRQRRFAGVVLAGAALGVATAAQITAGIALHQSNLNAQAIQSLRTSLEQSNKAIEEIREATQETIIAVQGVQDYVNNELVPAMQHMSCELVGQRLGLKLLRYYTELLSIFGPSLRDPISAEISIQALSYALGGEIHKILEKLGYSGNDMIAILESRGIKTKITHVDIPGKLIILSISYPTLSEVKGVIVHRLEAVSYNIGSQEWYTTVPRYVATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGVTIQVGGRQYPDMVYESKVALGPAISLERLDVGTNLGNALKKLDDAKILIDSSNQILETVRRSSFNFGSLLSVPILICTALALLFLIYCCKRR

SEQ ID NO: 24 - protein, 243 aa, synthetic construct
DIVLTQSPASLAVSLGQRATISCKASQSVDYDGDSYMNWYQQKPGQPPKLLIYAASNLESGIPDRFSGSGSGTDFIFNIHPVEEEDAATYFCQQFNEDPPTFGGGTKLEIKGGGGSGGGGSGGGGSQIQLQQSGPELVKPGASVKMSCKASGYTFTDYVIHWVKQRTGQGLEWIGEIYPGSGSSYYNENLKGKATLTADKSSNTVYILLSSLTSEDSAVYFCARRKVQGFAYWGQGTLVTVSA

SEQ ID NO: 25 - protein, 241 aa, synthetic construct
DVQITQSPSYLAASPGETITINCRASKSISKYLAWYQEKPGKTKKFLIYFGSTLQSGIPSRFSGSGSGTDFTLTISSLEPEDFAMYYCQQHDEYPLTFGAGTKLEIKGGGGSGGGGSGGGGSQVQLQQSGPELVRPGVSVKISCKGSGYTFTDYAMHWVKQSHAKSLEWIGIISTYYGNTKYNQKFKGKATMTVDKSSSTAYMELARLTSEDSAIYYCARKGDGNGAMDSWGQGTSVTVSS